(19) United States
(12) Patent Application Publication
(10) Pub. No.: US 2023/0132508 A1
(43) Pub. Date: May 4, 2023

(54) Uses of Tau Phosphosites as Biomarkers for Alzheimer's Disease

(71) Applicants: Genentech Inc., South San Francisco, CA (US); Washington University, St. Louis, MO (US)

(72) Inventors: Kristin Ruth Wildsmith, Half Moon Bay, CA (US); Balazs Toth, Foster City, CA (US); Randall Bateman, St. Louis, MO (US); Nicolas Barthelemy, St. Louis, MO (US)

(73) Assignees: Genentech Inc., South San Francisco, CA (US); Washington University, St. Louis, MO (US)

(21) Appl. No.: 17/914,817

(22) PCT Filed: Mar. 31, 2021
(86) PCT No.: PCT/US2021/025227

§ 371 Date: Sep. 27, 2022

Related U.S. Application Data
(60) Provisional application No. 63/119,882, filed on Dec. 1, 2020.
(60) Provisional application No. 63/003,781, filed on Apr. 1, 2020.

(51) Int. Cl. G01N 33/68

(52) U.S. Cl. CPC G01N 33/6896; G01N 2800/2821; G01N 2440/14; G01N 2800/56

(57) ABSTRACT

The present disclosure relates to methods of measuring and analyzing phosphorylation sites in the tau protein and determining correlations with other biomarkers of Alzheimer's disease and tauopathies.

### FIELD OF THE INVENTION

The present disclosure relates to methods of measuring and analyzing phosphorylation sites in the tau protein and determining correlations with other biomarkers of Alzheimer's disease and tauopathies.

### BACKGROUND

Alzheimer's disease (AD) is the most common cause of dementia, affecting an estimated 5.8 million individuals in the United States and 50 million worldwide (1). AD is characterized by the accumulation of extracellular beta-amyloid (Aβ) peptide-containing plaques (amyloid plaques) and intracellular tau aggregates (neurofibrillary tangles; NFTs) in the brain. Tau is a microtubule-binding protein that becomes hyperphosphorylated, and aggregates to form the primary constituent of NFTs. Post-mortem studies indicate that NFT density correlates more closely with neurodegeneration and cognitive impairment than A3 plaque density (41). NFTs within neuronal cell bodies are composed primarily of hyperphosphorylated tau (42).

Tau has over 80 potential phosphorylation sites (43), each with different influences on tau metabolism, conformation, and aggregation (44). In AD, sequential alteration of tau phosphorylation epitopes likely precedes aggregation and formation of tau filaments (45).

Tau is an attractive target for treatment of AD, since the anatomical distribution and extent of tau pathology correlate strongly with disease course and severity, i.e., with the scope of cognitive deficits and degree of cortical atrophy (2-6). Furthermore, in an AD transgenic (Tg) mouse model, knockout of the gene encoding tau (MAPT) protects against cognitive deficits (7), indicating tau may play a central role in the progression of the disease.

While characterized as a cytosolic protein, tau is reported to be found extracellularly under normal physiological conditions in brain interstitial fluid (ISF), cerebrospinal fluid (CSF), and plasma of rodents, nonhuman primates (NHPs), and humans, as well as in conditioned media of cultured neurons (8-16). Although the mechanism of tau excretion has not been fully elucidated, it is hypothesized that in the context of AD and possibly other tauopathies, pathogenic tau species spread from cell to cell via the extracellular environment and propagate in a prion-like fashion (17-19). Recent preclinical studies with locally expressed or injected pathological tau seeds support this tau spreading hypothesis (20-23). Moreover, in patients with AD, the propagation of tau pathology follows neuronal network circuits, suggesting a trans-synaptic mechanism of transmission (2,24). Anti-tau immunotherapy seeks to intercept tau in the extracellular space in the brain and attenuate the cell-to-cell propagation of abnormal tau.

Phosphorylation and fragmentation of tau have been suggested to play a role in tau pathogenesis where fragmentation has been shown to promote secretion of tau (25), and recent studies suggest that disease specific strains of tau are formed (26), proposing that the underlying molecular mechanism differ between various tauopathies. Studies have indicated that fragments of tau are present in CSF (27-31), brain (30, 32-34) and vesicles (35).

Neuroimaging and cerebrospinal fluid (CSF) biomarkers related to AD neuropathology are increasingly used to aid clinical diagnosis and patient selection for therapeutic trials (46, 47). CSF total tau (t-tau) and phosphorylated tau (p-tau) levels, which reflect the circulating pool of soluble tau released by central nervous system cells (9), may enable earlier assessment of tau abnormalities prior to the deposition of NFTs in brain parenchyma (48, 49). Many different tau isoforms exist and can be monitored in CSF (50, 31); however, it is unclear how these soluble tau species reflect brain neuropathology. Clarifying the relationships between tau pathology using tau PET and CSF tau isoforms may identify alterations in tau metabolism that occur before and concurrently to tau aggregation.

The present disclosure describes a mass spectrometry (MS) based method to monitor up to 11 phosphorylation sites across multiple tau peptides within the mid-domain region of tau (40). Unmodified tau peptides are quantified to enable calculation of the ratio of phosphorylated to unphosphorylated tau (p-tau/u-tau), thereby quantifying changes in tau phosphorylation ratios independently from variations in CSF tau levels. This method can be used, among other things, to compare established markers (e.g., p-tau181) with novel sites in order to evaluate their potential as biomarkers of tau pathology.

Using this MS approach, we previously demonstrated that several p-tau/u-tau ratios are significantly modified in CSF from late-onset AD participants (40). In particular, MS demonstrated that tau phosphorylation on Threonine 217 (T217) was positively associated with abnormal amyloid status, as measured by PiB PET and MS CSF Aβ 42/40 ratio, in both pre-clinical and mild AD (51, 52). CSF p-tau217 levels are more accurate in predicting amyloid load than p-tau181, the most commonly monitored tau phosphorylated site (53, 54). Recent studies also report better performance of p-tau217 than p-tau181 in plasma for predicting amyloid load (55, 56), highlighting the importance of alternative p-tau sites for diagnosing AD. Longitudinal CSF analyses in dominantly inherited AD (DIAD) confirm that p-tau217 is more tightly associated than p-tau181 with structural, metabolic, neurodegenerative, and clinical markers of disease (57).

Tau PET uptake correlates with clinical severity and poorer cognitive performance (58-63). Tau PET measures suggest pathological tau begins to aggregate around the time of symptom onset in DIAD and late-onset AD (64-66). Correlations between tau PET and CSF t-tau and pTau181 are modest (67-71) suggesting that soluble CSF tau species and tau PET may be differentially related to pathology and clinical symptoms. Indeed, some phospho-tau species may be pathologically altered prior to NFT deposition.

There remains a need to understand the relationships between CSF tau phosphorylation and stages of AD and other measures of Tau in patients. This present disclosure meets that need and provides other benefits. This present disclosure describes relationships between CSF tau phosphorylation (measured by MS at up to 11 disease-associated phosphosites) and underlying NFT pathology (measured by tau PET) in two separate cohorts of participants that spanned a full range of AD severity. These analyses allowed us to evaluate the clinical utility of measuring site-specific phosphorylation by clarifying its association with Aβ PET imaging, tau ([¹⁸F]GTP1) PET imaging, and cognition in sporadic AD.

### SUMMARY OF THE INVENTION

The invention provides a method for determining whether a human subject is likely to have Alzheimer's disease (AD), comprising detecting the level of pT217 in a cerebrospinal fluid (CSF) sample from the subject and correlating the level of pT217 in the sample with a tau PET SUVR (standard uptake value rate) for the subject, such as a tau ([¹⁸F]GTP1) PET SUVR; and determining whether the subject is likely to have AD based on the level of the pT217 compared to the tau PET SUVR, wherein the level of pT217 positively correlates with tau PET SUVR.

In some embodiments, the method comprises detecting the level of at least one further tau phosphosite in a cerebrospinal fluid (CSF) sample from the subject comprising (a) one or more of pT111, pT153, and pT175; (b) one or both of pS214 and pT231; (c) one or more of pT111, pT153, pT175, pS199, pS208, pS214, and pT231; (d) four or more of pT111, pT153, pT181, pS208, and pT231; or (e) one or more of pS199, pT175, and pS202. In some embodiments, the method comprises determining whether the subject is likely to have AD based on the level of the pT217 compared to the tau PET SUVR, wherein the level of pT217 positively correlates with tau PET SUVR, and further based on the levels of (a) one or more of pT111, pT153, and pT175; (b) one or both of pS214 and pT231; (c) one or more of pT111, pT153, pT175, pS199, pS208, pS214, and pT231; (d) four or more of pT111, pT153, pT181, pS208, and pT231; or (e) one or more of pS199, pT175, and pS202, wherein levels of pT175 and pS202 decrease with increasing AD stage, levels of pT111, pT153, pT181, pS199, pS208, pS214, pT217 and pT231 are higher in AD subjects than in CN subjects, and levels of pS214 and pT231 are higher in prodromal AD subjects than in CN subjects but lower in mild and moderate AD subjects than in prodromal AD subjects. In some embodiments, the method comprises determining whether the human subject is likely to have prodromal AD. In some embodiments, the method comprises determining whether the human subject is likely to have mild AD. In some embodiments, the method comprises determining whether the human subject is likely to have moderate AD.

In some embodiments, the method comprises staging a human subject as likely to be cognitively normal (CN) or to have prodromal, mild, or moderate Alzheimer's disease (AD), comprising: detecting a level of pT217 in a cerebrospinal fluid (CSF) sample from the subject and correlating the level of pT217 in the sample with a tau PET SUVR (standard uptake value rate) for the subject, such as a tau ([¹⁸F]GTP1) PET SUVR; and determining whether the subject is likely to have AD based on the level of the pT217 compared to the tau PET SUVR, wherein the level of pT217 positively correlates with tau PET SUVR.

In some embodiments, the method comprises detecting the level of at least one further tau phosphosite in a cerebrospinal fluid (CSF) sample from the subject comprising (a) one or more of pT111, pT153, and pT175; (b) one or both of pS214 and pT231; (c) one or more of pT111, pT153, pT175, pS199, pS208, pS214, and pT231; (d) four or more of pT111, pT153, pT181, pS208, and pT231; or (e) one or more of pS199, pT175, and pS202.

In some embodiments, the method comprises determining whether the subject is likely to have AD based on the level of the pT217 compared to the tau PET SUVR, wherein the level of pT217 positively correlates with tau PET SUVR, and further based on the levels of (a) one or more of pT111, pT153, and pT175; (b) one or both of pS214 and pT231; (c) one or more of pT111, pT153, pT175, pS199, pS208, pS214, and pT231; (d) four or more of pT111, pT153, pT181, pS208, and pT231; or (e) one or more of pS199, pT175, and pS202, wherein levels of pT175 and pS202 decrease with increasing AD stage, levels of pT111, pT153, pT181, pS199, pS208, pS214, pT217 and pT231 are higher in AD subjects than in CN subjects, and levels of pS214 and pT231 are higher in prodromal AD subjects than in CN subjects but lower in mild and moderate AD subjects than in prodromal AD subjects.

In some embodiments, the tau PET SUVR is tau ([¹⁸F]GTP1) PET SUVR. In some embodiments, the level of at least one of pS199, pT175, and pS202 is correlated with the tau PET SUVR, wherein each of pS199, pT175, and pS202 negatively correlates with tau PET SUVR.

In some embodiments, the method comprises determining whether a human subject is likely to have Alzheimer's disease (AD), comprising detecting the level of at least one tau phosphosite in a cerebrospinal fluid (CSF) sample from the subject comprising (a) one or more of pT111, pT153, and pT175, and optionally one or more of pT181, pS199, pS202, pT205, pS208, pS214, pT217, and pT231; (b) one or both of pS214 and pT231 and optionally one or more of pT111, pT153, pT175, pT181, pS199, pS202, pT205, pS208, and pT217; (c) pT217 and one or more of pT111, pT153, pT175, pS199, pS208, pS214, and pT231; or (d) four or more of pT111, pT153, pT181, pS208, pT217, and pT231; and determining whether the subject is likely to have AD based on the level of the at least one tau phosphosite in the sample compared to levels of the phosphosite in control samples of prodromal AD, mild AD, moderate AD, and/or cognitively normal (CN) subjects, wherein the levels of pT175 and pS202 decrease with increasing AD stage, the levels of pT111, pT153, pT181, pS199, pS208, pS214, pT217 and pT231 are higher in AD subjects than in CN subjects, and the levels of pS214 and pT231 are higher in prodromal AD subjects than in CN subjects but lower in mild and moderate AD subjects than in prodromal AD subjects. In some embodiments, the method comprises determining whether the human subject is likely to have prodromal AD. In some embodiments, the method comprises determining whether the human subject is likely to have mild AD. In some embodiments, the method comprises determining whether the human subject is likely to have moderate AD.

In some embodiments, the method comprises staging an Alzheimer's disease (AD) or potential AD subject as likely to be clinically normal (CN) or to have prodromal, mild, or moderate AD, comprising: detecting the level of at least one tau phosphosite in a CSF sample from the subject comprising (a) one or more of pT111, pT153, and pT175, and optionally one or more of pT181, pS199, pS202, pT205, pS208, pS214, pT217, and pT231; (b) one or both of pS214 and pT231 and optionally one or more of pT111, pT153, pT175, pT181, pS199, pS202, pT205, pS208, and pT217; (c) pT217 and one or more of pT111, pT153, pT175, pS199, pS208, pS214, and pT231; or (d) four or more of pT111, pT153, pT181, pS208, pT217, and pT231; and determining whether the subject is likely to have AD based on the level of the at least one tau phosphosite in the sample compared to levels of the phosphosite in control samples of prodromal AD, mild AD, moderate AD, and/or cognitively normal (CN) subjects, wherein levels of pT175 and pS202 decrease with increasing AD stage, levels of pT111, pT153, pT181, pS199, pS208, pS214, pT217 and pT231 are higher in AD subjects than in CN subjects, and levels of pS214 and pT231 are higher in prodromal AD subjects than in CN subjects but lower in mild and moderate AD subjects than in prodromal AD subjects.

In some embodiments, the method comprises correlating the level of the at least one tau phosphosite in the sample with a tau PET SUVR (standard uptake value rate) for the subject, such as a tau ([¹⁸F]GTP1) PET SUVR. In some embodiments, the level of at least one of pT217, pS199, pT175, and pS202 is correlated with the tau PET SUVR, wherein pT217 positively correlates with tau PET SUVR, and wherein each of pS199, pT175, and pS202 negatively correlates with tau PET SUVR. In some embodiments, the subject is known to be A3+, for example, based on an amyloid beta PET scan; has not received an amyloid beta PET scan; has not previously received a tau PET scan, such as a tau ([18F]GTP1) PET imaging scan; or has not previously received either an amyloid beta PET scan or a tau PET scan. In some embodiments, the level of at least one tau phosphosite in a CSF sample from the subject is detected by liquid chromatography mass spectrometry (e.g. tandem mass spectrometry) analysis (LC-MS or LC-MS/MS)). In some embodiments, the CN subjects comprise Aβ+ but CN subjects, Aβ− CN subjects, or all CN subjects regardless of Aβ status.

In some embodiments, the method comprises detecting the level of one or more of pT111, pT153, or pT175. In some embodiments, the method comprises determining whether the level of pT111 and/or pT153 is elevated in comparison to CN subjects, and/or wherein the method comprises determining whether the level of pT175 is reduced in comparison to CN subjects and/or in comparison to prodromal or mild AD subjects. In some embodiments, the method comprises detecting the level of two or more of pT111, pT153, pT205, pS208, pS199, and pT217. In some embodiments, the method comprises detecting the level of one or more of pT181, pS214, and pT231, and determining whether the level of pT181, pS214, and/or pT231 is elevated in comparison to CN subjects or is elevated in comparison to mild or moderate AD subjects. In some embodiments, the method comprises detecting the level of two or more of pT181, pS214, and pT231, or all three of pT181, pS214, and pT231. In some embodiments, the method comprises method comprises detecting the level of pS214, and determining whether the level of pS214 is elevated in comparison to CN subjects or is elevated in comparison to mild or moderate AD subjects. In some embodiments, the method comprises detecting the level of one or both of pT175 and pS202, and determining whether the level of pT175 and/or pS202 is reduced in comparison to CN subjects and/or is elevated in comparison to mild or moderate AD subjects. In some embodiments, the method comprises detecting the level of no more than 11 phosphosites. In some embodiments, the method comprises detecting the level of no more than 8 phosphosites. In some embodiments, the method comprises detecting the level of no more than 6 phosphosites. In some embodiments, the method comprises detecting the level of no more than 4 phosphosites. In some embodiments, the method comprises detecting the level of one or more of pT111, pT153, and pT175, and additionally one or more of pT181, pS199, pS202, pT205, pS208, pS214, pT217, and pT231. In some embodiments, the method comprises detecting the level of one or both of pT175 and pS202. In some embodiments, the method comprises detecting the level of one or both of pS214 and pT231 and additionally one or more of pT111, pT153, pT175, pT181, pS199, pS202, pT205, pS208, and pT217. In some embodiments, the method comprises detecting the level of pT217 and one or more of pT111, pT153, pT175, pS199, pS208, pS214, and pT231. In some embodiments, the method comprises detecting the level of each of pT217, pT153, pT181, pT111, pS208, and pT231.

In some embodiments, the method comprises the level of the phosphosite(s) is calculated as the ratio of the level of the phosphorylated site to the level of its corresponding unposphorylated site (e.g., by calculating the ratio of the level of a peptide comprising the phosphorylated site to the level of the corresponding unphosphorylated peptide in a MS analysis). In some embodiments, the subject has not previously had a tau PET and/or amyloid beta PET and wherein the method is used to determine whether the subject should receive a tau PET and/or amyloid beta PET. In some embodiments, the method comprises performing one or more of tau PET, amyloid PET, MRI, pTau181 ELISA, and cognitive function assays on the subject.

In some embodiments, the method comprises treating a subject with an anti-tau therapy, wherein the subject has been tested prior to treatment according to a method described herein and found to have a level of tau phosphosite(s) corresponding to that of an AD subject (e.g., a prodromal, mild, or moderate AD subject). In some embodiments, the method comprises treating a subject with an anti-tau therapy, wherein the subject has been tested prior to treatment according to a method described herein, the method comprising determining that the subject has a level of tau phosphosite(s) corresponding to that of an AD subject (e.g., a prodromal, mild, or moderate AD subject), and subsequently administering an anti-tau therapy to the subject. In some embodiments, the anti-tau therapy comprises an anti-tau antibody. In some embodiments, the anti-tau antibody is gosuranemab, ABBV-8E12, zagotenemab, or semorinemab. In some embodiments, the anti-tau antibody is semorinemab.

In some embodiments, the subject is re-tested according to the methods described herein following administration of the anti-tau therapy.

### BRIEF DESCRIPTION OF THE FIGURES

Color versions of the figures herein were provided with the priority applications, which are incorporated by reference herein.

FIG. 1A shows how tau pathology correlates with cognition and spreads with increasing disease severity (MMSE scores and Tau tangle count).

FIG. 1B shows tau present in a neurofibrillary tangle. Hyperphosphorylated tau is one of the major components of tangles in Alzheimer's disease.

FIG. 2 shows a depiction of how tau pathology may spread in a prion-like manner.

FIG. 3 shows a depiction of ways to measure tau using positron emission tomography (PET) and cerebral spinal fluid (CSF)-based methods.

FIGS. 4A-B show data from tau PET assays using a [¹⁸F]GTP1 (Genentech Tau Probe 1) PET tracer. FIG. 4A shows how [¹⁸F]GTP1 increases with disease severity in AD, and is selective for tau pathology. FIG. 4B shows regions of interest for tau PET SUVR analyses in temporal meta (TMP) and whole cortical grey matter (WCG) and Braak-related regions.

FIG. 5A shows phosphorylation sites (or phosphosites) across the domains of the tau protein and percent (%) phosphorylation at those sites (red=hyperphosphorylated; blue=reference; green=hypophosphorylated) in brain soluble CSF, non-AD CSF, and AD CSF measured by multiplexed IP-LC/MS. CSF Tau was also measured by an Elecsys® pTau181 assay (area shaded in purple, encompassing residues from about 175 to 231). In the non-AD CSF diagram, position 181 is colored in blue, position 202 in green (hypophosphorylated), and positions 111, 205, 208, 217, and 231 in red (hyperphosphorylated). In the AD CSF diagram, positions 181 and 231 are colored in lighter red (less hyperphosphorylated), position 202 is in lighter green (less hypophosphorylated), while positions 111, 205, 208, and 217 remain in red (hyperphosphorylated). As also shown in the non-AD CSF and AD CSF diagrams, the sizes of the circles corresponding to residues such as 111, 205, and 217 also differ, further indicating differences in the degree of phosphorylation.

FIG. 5B shows that phosphorylation at multiple sites on the tau protein can be detected. CSF Tau was measured by multiplexed IP-LC/MS and relative intensities of detectable peaks at 11 phosphosites are depicted.

FIG. 6 shows analysis of CSF samples from participants in Alzheimer's disease trials with paired [¹⁸F]GTP1.

FIG. 7 shows that [¹⁸F]GTP1 SUVR increases with disease severity in the broad cohort.

FIGS. 8A-F show that CSF pTau181 (Elecsys®) increases in early AD and plateaus. FIGS. 8A-B show data in pg/mL from two studies NHS (FIG. 8A) and Tauriel (FIG. 8B). A summary of the data is provided in FIG. 8E. FIGS. 8C-D show data in a ratio of phosphorylated Tau 181 (pTau 181) to total tau (tTau) from the same studies (FIG. 8C; NHS and FIG. 8D; Tauriel). A summary of the data is provided in FIG. 8F. CSF pTau levels are shown for CN−, CN+, Prodromal, Mild, and Moderate AD groups.

FIGS. 9A-O show cross-sectional boxplots from the full range AD cohort comparing brain amyloid deposition measured by florbetapir (FBP) tracer (FIG. 9A), [¹⁸F]GTP1 SUVR measuring brain tau aggregates (FIG. 9B), p-tau measures by Elecsys® pTau Immunoassay on T181 (FIG. 9C, levels I (pg/mL); ratio ptau/tau (FIG. 9D), tau phosphorylation ratios (ptau/utau) at 181 by MS (FIG. 9E), and on other phosphorylated residues by MS: pT111 (FIG. 9F), pT153 (FIG. 9G), pT175 (FIG. 9H), pS199 (FIG. 9I), pS202 (FIG. 9J), pT205 (FIG. 9K), pS208 (FIG. 9L), pS214 (FIG. 9M), pT217 (FIG. 9N), and pT231 (FIG. 9O). In all figures, CN−, amyloid-negative control; CN+, amyloid-positive control; prod, prodromal; mod, moderate.

FIGS. 10A-O show scatter plots from the early AD cohort comparing MMSE and brain amyloid deposition measured by FBP tracer (FIG. 10A), [¹⁸F]GTP1 SUVR measuring brain tau aggregates (FIG. 10B), p-tau measures by Elecsys® pTau Immunoassay on p-tau181 in pg/mL (FIG. 10C) and as p-tau/total tau ratio by Elecsys® (FIG. 10D), tau phosphorylation occupancy on 181 (p-tau/total tau ratio) by MS (FIG. 10E), and on other phosphorylated residues monitored by MS (FIG. 10F-O), specifically: pT111 (FIG. 10F), pT153 (FIG. 10G), pT175 (FIG. 10H), pS199 (FIG. 10I), pS202 (FIG. 10J), pT205 (FIG. 10K), pS208 (FIG. 10L), pS214 (FIG. 10M), pT217 (FIG. 10N), and pT231 (FIG. 10O). The grey curve and the shaded area show the locally weighted average phosphorylation and its 95% confidence interval. In FIG. 10A, x, FBB; circle with hash, FBP. In FIGS. 10B-10O, filled circles, prodromal; filled boxes, mild.

FIGS. 11A-D show cross-correlation between tau phosphorylation ratios on monitored sites in the full range AD cohort (FIGS. 11A-C) and early AD cohort (FIG. 11D). The two cohorts support the identification of independent groups of association on site-specific phosphorylation occupancies that shift with disease severity (FIG. 11A—full range AD cohort; FIG. 11B—full range AD cohort CN-prodromal patients; FIG. 11C—full range AD cohort mild-moderate patients; FIG. 11D—early AD cohort). The dendograms represent clusters of CSF hyperphosphorylation at different sites and resemble the pattern similarities as seen in FIGS. 9A-O.

FIGS. 12A-B show association between [¹⁸F]GTP1 SUVR and CSF p-tau measures. Correlation comparison (Spearman r) between measures of amyloid PET SUVR (FBP or FBB) and brain tau aggregation in various regions of interest using [¹⁸F]GTP1 tracer (tmp_metaROI, WCG, Braak12, Braak34, Braak56) with CSF p-tau measures and amyloid PET SUVR in full range AD (FIG. 12A) and early AD (FIG. 12B).

FIGS. 13A-D show association between [¹⁸F]GTP1 SUVR and CSF p-tau measures. Correlation between [¹⁸F]GTP1 SUVR (tmp_metaROI) and CSF tau phosphorylation occupancy on T217 in the full range AD cohort (including cognitively normal amyloid-negative and -positive, prodromal, mild and moderate AD, FIG. 13A) and early AD cohort (prodromal and mild AD, FIG. 13B). Correlation between A3 PET SUVR and CSF tau phosphorylation occupancy on T217 in the full range AD cohort (FIG. 13C) and early AD cohort (FIG. 13D). As shown adjacent to FIG. 13A, CN− amyloid-negative controls are depicted by open, downward triangles, CN+ amyloid-positive controls by filled upward triangles, prodromal (prod) by filled circles, mild by filled squares, and moderate (mod) by filled diamonds. Prodromal AD curves are provided in each sub-figure and are the higher sloped curves in FIGS. 13A-C and the lower of the two curves in FIG. 13D; mild AD curves are also shown in each sub-figure and are the next highest sloped curves in FIGS. 13A-C and the upper of the two curves in FIG. 13D; moderate AD curves are shown in FIGS. 13A and 13C and have a lower slope than the prodromal and mild AD curves. Curves corresponding to CN− and CN+ subjects are to the lower left of FIGS. 13A and 13C with empty triangles showing CN− and filled triangles CN+.

FIGS. 14A-F show that pT217/T217 offers a potential greater differentiation between controls and AD and may continue to rise through mild AD. FIG. 14A shows the phosphorylated Tau to total Tau (pTau/tTau) ratio at 181 (measured in the Elecsys® assay) for clinically normal CN− and CN+ controls and prodromal, mild, and moderate AD subjects in the NHS study, while FIG. 14C shows the same data for the Tauriel study. FIG. 14B shows the ratio of p217 to total 217 for clinically normal CN− and CN+ controls and prodromal, mild, and moderate AD subjects in the NHS study, while FIG. 14D shows the same data for the Tauriel study. FIGS. 14E and 14F show summaries of the pTau/tTau 181 and p217/t217 data in chart format, respectively.

FIGS. 15A-C show that the pT217/tTau correlation has the highest correlation with Amyloid PET. FIG. 15A and FIG. 15B show how pT217/T217 correlate with Amyloid PET in two different studies. In FIG. 15A, Amyloid (−) subjects are shown as a cluster of closed circles near the zero point on the X axis, and from about zero to 30% on the Y axis, while Amyloid (+) subjects are shown with closed circles from about 0.2 to 1.3 on the X axis. In FIG. 15B, clinically normal CN− and CN+ subjects are represented with open downward and closed upward triangles, while prodromal, mild, and moderate AD subjects are represented with circles, squares, and diamonds, respectively. FIG. 15C provides Spearman correlations based on the data.

FIGS. 16A-B show forest plots of 4 different cognitive assessments (MMSE, CDR-SB, ADAS13, and RBANS_TOTAL) that were performed for several phosphosites for the NHS (FIG. 16A) and Tauriel studies (FIG. 16B). MMSE, CDR-SB, ADAS13, and RBANS_TOTAL are represented by diamonds, upward triangles, squares, and circles, respectively, with closed marks representing the most significant Spearman correlations (p≤0.05) and open marks representing less significant Spearman correlations (p>0.05).

FIG. 17 describes that pT217/T217 correlated most strongly with [¹⁸F]GTP1 in both cohorts, including Elecsys® pTau181/tTau.

FIGS. 18A-B show association measured by Spearman r between [¹⁸F]GTP1 and CSF p-tau measures with brain atrophy as measured by MRI in different regions in the full range cohort, excluding cognitively normal individuals (FIG. 18A), and in the early AD cohort (FIG. 18B).

FIGS. 19A-C show association between [¹⁸F]GTP1 SUVR (tmp_metaROI) and CSF p-tau measures with cognitive scores in different regions in the full range AD cohort (FIG. 19A), in the full range AD cohort excluding cognitively normal individuals (FIG. 19B) and in the early AD cohort (FIG. 19C).

### DETAILED DESCRIPTION OF THE INVENTION

This disclosure, inter alia, provides methods for determining the phosphorylation levels and phosphorylation ratios of phosphosites in tau proteins and methods for determining which tau phosphosites in cerebrospinal fluid (“CSF”) correlate with a tau-based PET tracer such as [¹⁸F]GTP1 PET ([¹⁸F]GTP1 PET is also known as “Genentech tau probe 1”) in Alzheimer's disease (AD) using an expanded phospho-tau LC-MS/MS assay.

In some aspects, this disclosure provides tau phosphosites or sets of tau phosphosites detectable in biological fluids of a human subject that may be used in determining, for example, stage of a tauopathy such as AD in the subject, in some cases in addition to or as an alternative to existing biomarker assays such as tau-based PET tracer (“tau-PET”) assays. In some aspects, this disclosure provides tau phosphosites or sets of tau phosphosites that may be used in selecting human subjects suitable for treatment with anti-tau therapies, such as anti-tau antibodies, and/or for predicting human subjects that may be responsive to anti-tau therapies, such as anti-tau antibodies.

In some aspects, this disclosure provides a method for determining whether a human subject is likely to have prodromal, mild, and/or moderate AD based on levels of certain tau phosphosites in a CSF sample from the subject. In some aspects, this disclosure provides a method for determining whether a human subject is likely to have prodromal, mild, and/or moderate AD as opposed to no AD symptoms. In some aspects, the subject is known to be amyloid beta positive (Aβ+), for example, based on an amyloid beta PET scan. In other aspects, an amyloid beta PET scan has not been run on the subject. In some aspects, the method is for determining whether the subject is likely to have prodromal AD as opposed to mild and/or moderate AD. In some aspects, the method is for determining whether the subject is likely to have mild AD as opposed to prodromal and/or moderate AD. In some aspects, the method is for determining whether the subject is likely to have moderate AD as opposed to mild and/or prodromal AD.

In some aspects, the method comprises detecting the level of one or more of the following tau phosphosites in a biological sample from the subject: pT111, pT153, pT205, pS208, pS199, pT217, pT181, pS214, pT231, pT175, and/or pS202. In some aspects, the sample is a CSF sample. In some aspects, the method comprises determining the level of one or more of pT111, pT153, pT205, pS208, pS199, pT217, pT181, pS214, pT231, pT175, and/or pS202 in comparison to its level in subjects who are Aβ+ but cognitively normal (CN). In some aspects, the method comprises determining the level of one or more of pT111, pT153, pT205, pS208, pS199, pT217, pT181, pS214, pT231, pT175, and/or pS202 in comparison to its level in subjects who are either cognitively normal (CN) regardless of Aβ status or who are CN and also Aβ negative (Aβ−). In some aspects, the method comprises determining the level of at least two of pT111, pT153, pT205, pS208, pS199, pT217, pT181, pS214, pT231, pT175, and pS202. In some aspects, determining the level of the tau phosphosite comprises determining the ratio of the level of the phosphorylated site to the level of the unphosphorylated site (e.g., pT217/T217) or alternatively, determining the ratio of the level of the phosphorylated site to the level of the site as a whole, phosphorylated plus unphosphorylated (e.g., pT217/total T217). Thus, the “level” as recited in this context is actually a ratio. These ratios could be determined, for example, by quantitating a MS peak for a phosphorylated peptide containing the phosphosite and also a MS peak for an unphosphorylated peptide containing the phosphosite, and then determining the ratios of the peptides as above.

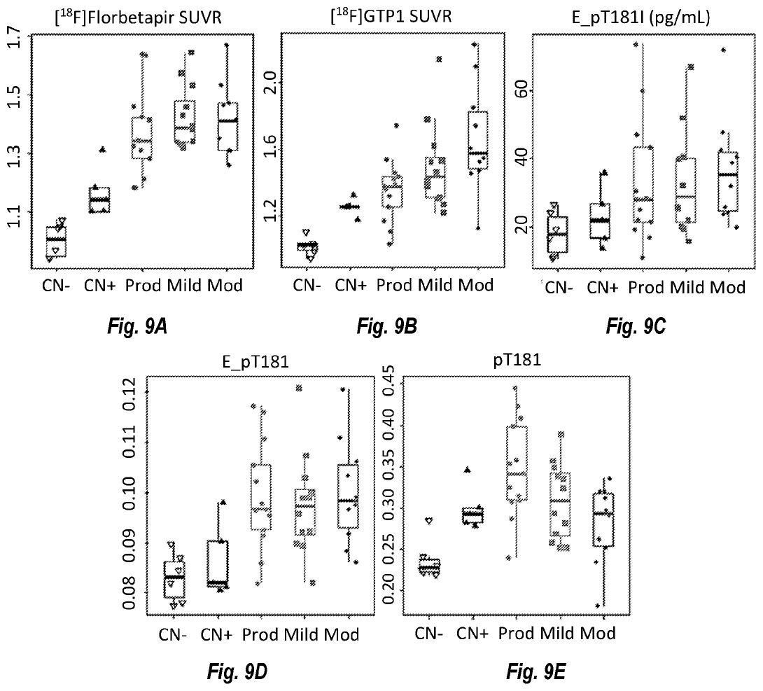

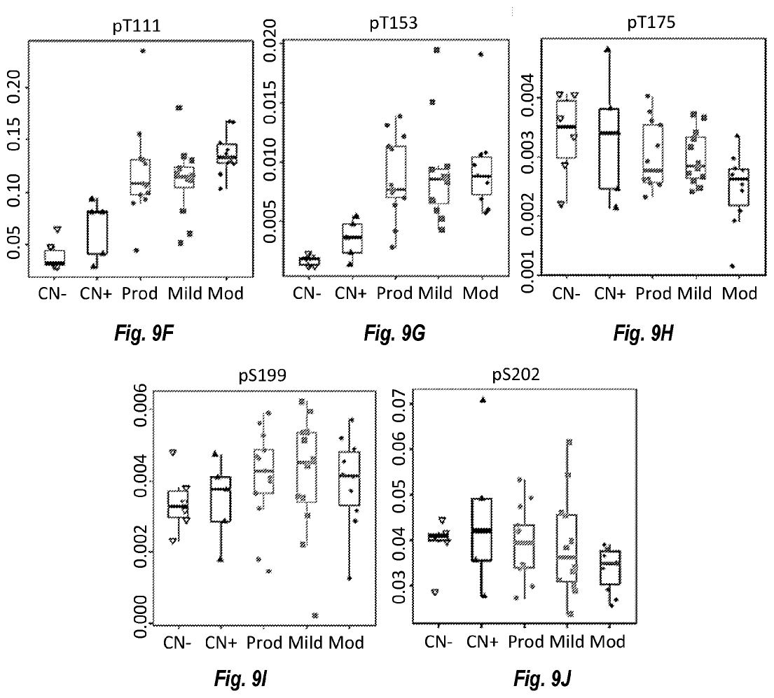

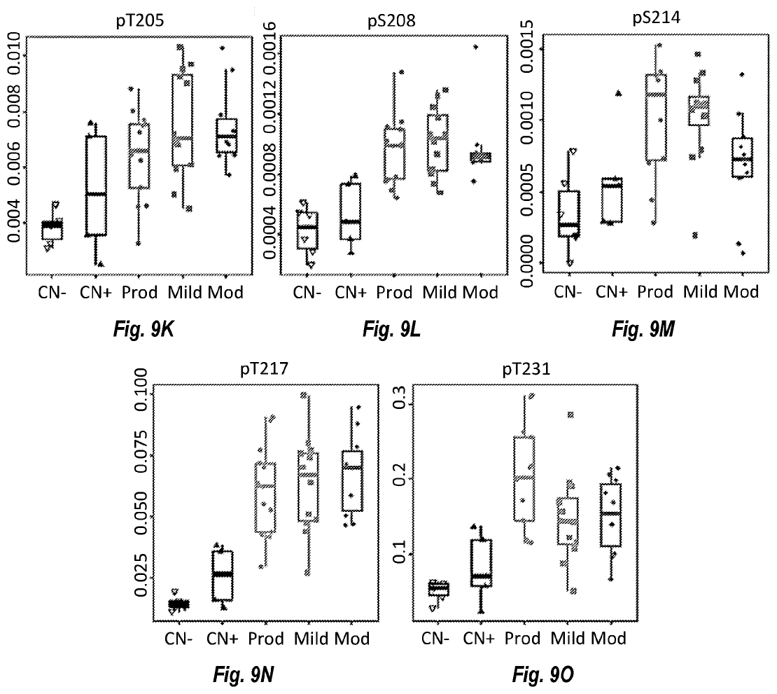

In some aspects, the method comprises detecting the level of one or more of the following tau phosphosites in a biological sample from the subject: pT111, pT153, pT205, pS208, pS199, and/or pT217. As noted again below, levels of these tau phosphosites (measured as ratios of phosphorylated site level over total site level, e.g., pT217/T217) in CSF samples were found to be elevated in human subjects with prodromal, mild or moderate AD in comparison to clinically normal subjects, but not to significantly differ among the prodromal, mild, or moderate subjects studied. (See FIGS. 9A-O.) In some aspects, the sample is a CSF sample. In some aspects, the method comprises determining whether the level of one or more of pT111, pT153, pT205, pS208, pS199, and/or pT217 is elevated in comparison to its level in subjects who are Aβ+ but cognitively normal (CN). In some aspects, the method comprises determining whether the level of one or more of pT111, pT153, pT205, pS208, pS199, and/or pT217 is elevated in comparison to its level in subjects who are either cognitively normal (CN) regardless of Aβ status or who are CN and also Aβ negative (Aβ−). In some aspects, the method comprises determining the level of at least two of pT111, pT153, pT205, pS208, pS199, pT217, pT181, pS214, pT231, pT175, and pS202. In some aspects, the level of the tau phosphosite is determined as a ratio of the level of the phosphorylated site to the level of the unphosphorylated site (e.g., pT217/T217) or alternatively, as a ratio of the level of the phosphorylated site to the level of the site as a whole, phosphorylated plus unphosphorylated (e.g., pT217/total T217).

In some aspects, the method comprises detecting the level of one or more of the following tau phosphosites in a biological sample from the subject: pT181, pS214, and/or pT231. In some aspects, the sample is a CSF sample. As noted below, levels of these three tau phosphosites were found to be elevated in prodromal, mild, and moderate AD subjects compared with CN subjects, but also to have higher levels in prodromal AD subjects compared to mild AD subjects and higher levels in mild AD subjects compared to moderate AD subjects. (See FIGS. 9A-O.) In some aspects, the method comprises determining whether the level of one or more of pT181, pS214, and/or pT231 is elevated in comparison to its level in subjects who are Aβ+ but cognitively normal (CN). In some aspects, the method comprises determining whether the level of one or more of pT181, pS214, and/or pT231 is elevated in comparison to its level in subjects who are either cognitively normal (CN) regardless of Aβ status or who are CN and also Aβ negative (Aβ−). In some aspects, the method comprises determining how the level of pT181, pS214, and/or pT231 in the subject compares to an expected level for a prodromal, mild, and/or moderate AD subject. In some aspects, the method comprises determining the level of at least two of pT181, pS214, and/or pT231. In some aspects, the method comprises determining the level of all three of pT181, pS214, and pT231. In some aspects, the method comprises determining the level of pS214 and/or pT231. In some aspects, the level of the tau phosphosite is determined as a ratio of the level of the phosphorylated site to the level of the unphosphorylated site (e.g., pS214/5214) or alternatively, as a ratio of the level of the phosphorylated site to the level of the site as a whole, phosphorylated plus unphosphorylated (e.g., pS214/total 5214).

In some aspects, the method comprises detecting the level of one or more of the following tau phosphosites in a biological sample from the subject: pT175 and/or pS202. In some aspects, the sample is a CSF sample. As noted below, levels of these two phosphosites were found to be reduced in prodromal, mild, and moderate AD subjects compared to CN subjects, with moderate AD subjects having lower levels than mild AD subjects and mild AD subjects showing lower levels than prodromal AD subjects. (See FIGS. 9A-O.) In some aspects, the method comprises determining whether the level of pT175 and/or pS202 is reduced in comparison to its level in subjects who are Aβ+ but cognitively normal (CN). In some aspects, the method comprises determining whether the level of pT175 and/or pS202 is reduced in comparison to its level in subjects who are either cognitively normal (CN) regardless of Aβ status or who are CN and also Aβ negative (Aβ−). In some aspects, the method comprises determining how the level of pT175 and/or pS202 in the subject compares to an expected level for a prodromal, mild, and/or moderate AD subject. In some aspects, the method comprises determining the level of both pT175 and pS202. In some aspects, the level of the tau phosphosite is determined as a ratio of the level of the phosphorylated site to the level of the unphosphorylated site (e.g., pT175/T175) or alternatively, as a ratio of the level of the phosphorylated site to the level of the site as a whole, phosphorylated plus unphosphorylated (e.g., pT175/total T175).

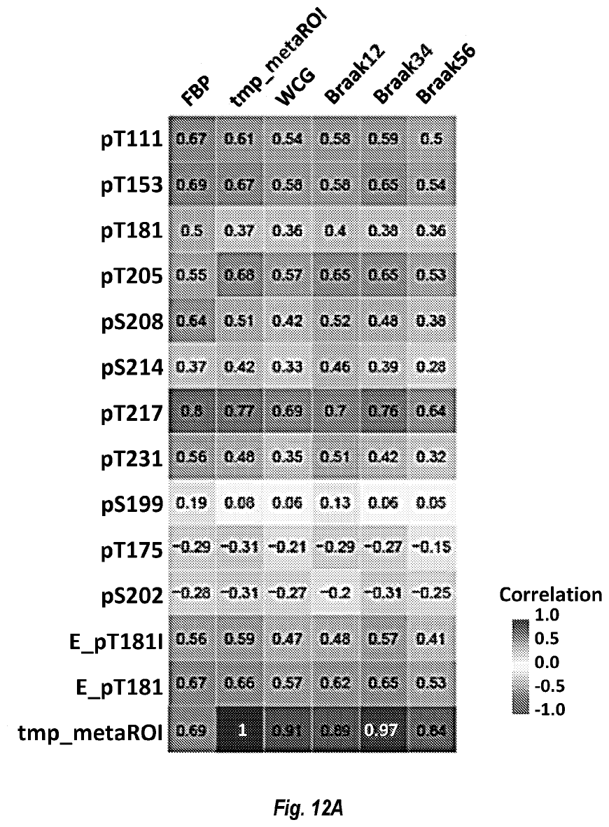

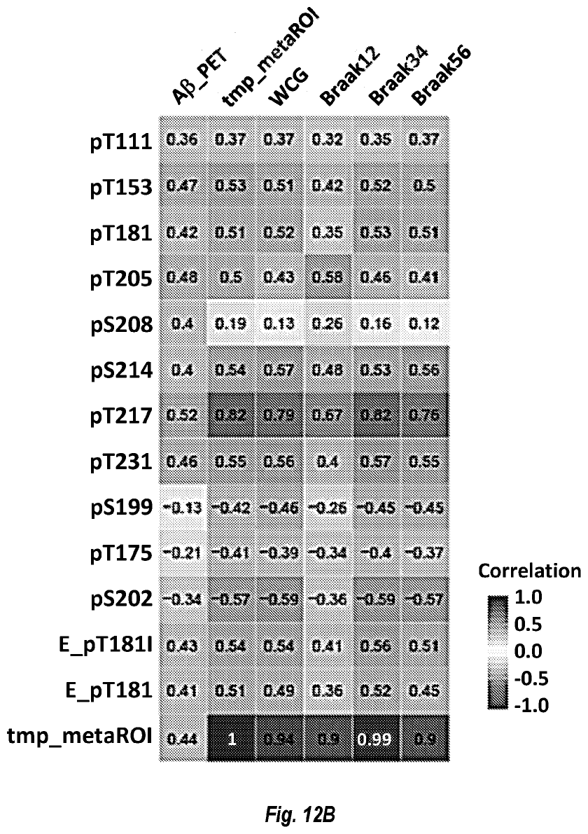

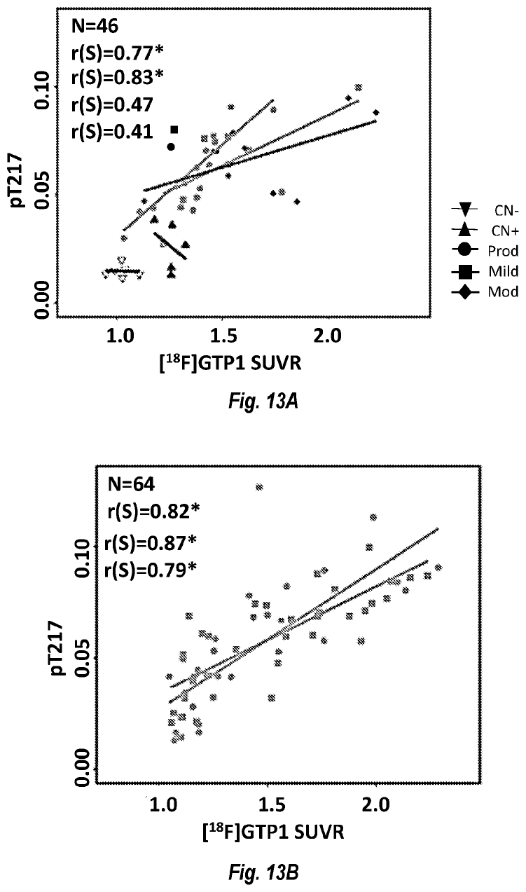

In some aspects, the method comprises comparing the level of at least one of pT111, pT153, pT205, pS208, pS199, and pT217 to that of cognitively normal (CN) subjects (e.g., either or both of Aβ− and Aβ+ CN subjects), and determining whether the level is increased relative to CN subjects. For example, as shown in FIGS. 9A-O, levels of each of these phosphosites tend to be higher in prodromal AD, mild AD, and moderate AD subjects compared to those in CN subjects (both Aβ− and Aβ+ CN subjects). In some aspects, the method comprises comparing the level of at least one of pT181, pS214, and pT231 to that of cognitively normal (CN) subjects (e.g., either or both of Aβ− and Aβ+ CN subjects), and determining whether the level is increased relative to CN subjects. For example, as shown in FIGS. 9A-O, levels of each of these phosphosites tend to be higher in prodromal AD subjects compared to those in CN subjects (both Aβ− and Aβ+ CN subjects). Levels of pT181, pS214, and pT231 also tended to spike in prodromal AD subjects (see FIGS. 9A-O) compared to mild and moderate AD subjects. Thus, in some embodiments, the method comprises comparing levels of one or more of those phosphosites to levels from prodromal, mild, and/or moderate AD subjects, for example, to help stage an AD or possible AD subject. In some aspects, the method comprises comparing the level of one or both of pT175 and pS202 to that of cognitively normal (CN) subjects (e.g., either or both of Aβ− and Aβ+ CN subjects), and determining whether the level is decreased relative to CN subjects. For example, as shown in FIGS. 9A-O, levels of each of these phosphosites tend to be lower in prodromal AD, mild AD, and moderate AD subjects compared to those in CN subjects (both Aβ− and Aβ+ CN subjects). In some embodiments, the method comprises correlating the level of at least one of pT217, pS199, pT175, and pS202 with that of a measurement obtained from a tau PET tracer (e.g. ¹⁸F GTP1 tau PET). As shown in FIGS. 12A-B, for example, pT217 positively correlates with values from an ¹⁸F GTP1 tau PET assay, while each of pS199, pT175, and pS202 negatively correlate with values from an ¹⁸F GTP1 tau PET assay.

In some aspects, at least one of each type of the above markers is selected for analysis. In some aspects, the method comprises detecting the level of one or more of the following tau phosphosites in a biological sample from the subject: (a) at least one of pT111, pT153, pT205, pS208, pS199, and pT217, (b) at least one of pT181, pS214, and pT231, and/or (c) at least one of pT175 and pS202. In some aspects, the method comprises detecting the level of one or more of the following tau phosphosites in a biological sample from the subject: (a) at least one of pT111, pT153, pT205, pS208, pS199, and pT217, and (b) at least one of pT181, pS214, and pT231. In some aspects, the method comprises detecting the level of one or more of the following tau phosphosites in a biological sample from the subject: (b) at least one of pT181, pS214, and pT231, and (c) at least one of pT175 and pS202. In some aspects, the method comprises detecting the level of one or more of the following tau phosphosites in a biological sample from the subject: (a) at least one of pT111, pT153, pT205, pS208, pS199, and pT217, and (c) at least one of pT175 and pS202. In some aspects, the sample is a CSF sample. In some aspects, the levels are compared to those of CN subjects that are Aβ+, Aβ−, or regardless of Aβ status. In some aspects, the level of the tau phosphosite is determined as a ratio of the level of the phosphorylated site to the level of the unphosphorylated site (e.g., pT217/T217) or alternatively, as a ratio of the level of the phosphorylated site to the level of the site as a whole, phosphorylated plus unphosphorylated (e.g., pT217/total T217). In some embodiments, determining levels of more than one type of tau phosphosite marker may be helpful in distinguishing prodromal, mild, and moderate AD subjects, or in predicting likelihood that a subject has a prodromal, mild, or moderate pathology.

In some embodiments, one or more of the above methods may be used in addition to, or as an alternative to, a more invasive diagnostic method, such as PET. Thus, in any of the above methods, the subject in some aspects has not received PET scanning. In some aspects, the subject has not received tau-based PET scanning. In some aspects, the subject has not received amyloid beta-based PET scanning.

In some embodiments, where the subject has not received tau-based PET scanning or whether the method is used as an alternative to tau-based PET scanning, the method comprises detecting the level of one or more of pT217, pS199, pT175, or pS202 in a biological sample (e.g. a CSF sample) from the subject. In some embodiments, where the subject has not received tau-based PET scanning or whether the method is used as an alternative to tau-based PET scanning, the method comprises detecting the level of pT217 in a biological sample (e.g. a CSF sample) from the subject. In some embodiments, where the subject has not received tau-based PET scanning or whether the method is used as an alternative to tau-based PET scanning, the method comprises detecting the level of pS199 in a biological sample (e.g. a CSF sample) from the subject. In some embodiments, where the subject has not received tau-based PET scanning or whether the method is used as an alternative to tau-based PET scanning, the method comprises detecting the level of pT175 in a biological sample (e.g. a CSF sample) from the subject. In some embodiments, where the subject has not received tau-based PET scanning or whether the method is used as an alternative to tau-based PET scanning, the method comprises detecting the level of pS202 in a biological sample (e.g. a CSF sample) from the subject. In some embodiments, where the subject has not received tau-based PET scanning or whether the method is used as an alternative to tau-based PET scanning, the method comprises detecting the level of at least two of pT217, pS199, pT175, or pS202 in a biological sample (e.g. a CSF sample) from the subject, such as pT217 plus one or more of pS199, pT175, or pS202.

In some embodiments, where the subject has not received tau-based PET scanning or whether the method is used as an alternative to amyloid beta-based PET scanning, the method comprises detecting the level of one or more of pT217, pT175, or pS202 in a biological sample (e.g. a CSF sample) from the subject. In some embodiments, where the subject has not received Aβ-based PET scanning or where the method is used as an alternative to tau-based PET scanning, the method comprises detecting the level of pT217 in a biological sample (e.g. a CSF sample) from the subject. In some embodiments, where the subject has not received Aβ-based PET scanning or where the method is used as an alternative to Aβ-based PET scanning, the method comprises detecting the level of pT175 in a biological sample (e.g. a CSF sample) from the subject. In some embodiments, where the subject has not received tau-based PET scanning or where the method is used as an alternative to Aβ-based PET scanning, the method comprises detecting the level of pS202 in a biological sample (e.g. a CSF sample) from the subject. In some embodiments, where the subject has not received Aβ-based PET scanning or where the method is used as an alternative to tau-based PET scanning, the method comprises detecting the level of at least two of pT217, pT175, or pS202 in a biological sample (e.g. a CSF sample) from the subject, such as pT217 plus one or both of pT175 and pS202.

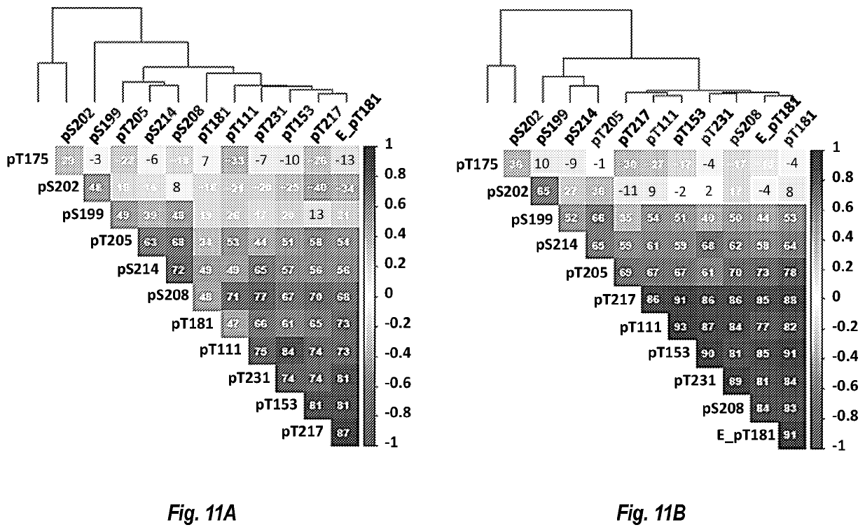

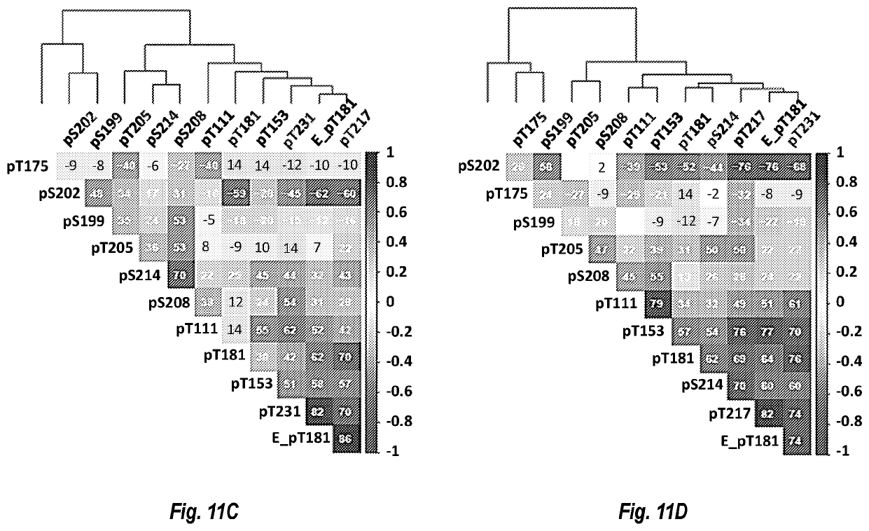

In some aspects, where the subject has not received tau-based PET scanning or whether the method is used as an alternative to amyloid beta-based PET scanning, the method comprises comparing the level of one or both of pT175 and pS202 to that of cognitively normal (CN) subjects (e.g., either or both of Aβ− and Aβ+ CN subjects), and determining whether the level is decreased relative to CN subjects. For example, as shown in FIGS. 9A-O, levels of each of these phosphosites tend to be lower in prodromal AD, mild AD, and moderate AD subjects compared to those in CN subjects (both Aβ− and Aβ+ CN subjects). In some embodiments, the method comprises correlating the level of at least one of pS199, pT175, and pS202 with that of pT217. As shown in FIGS. 11A-D and 12A-B, for example, pS199, pT175, and pS202 negatively correlate with pT217.

In other embodiments, one or more of the above methods may be used to determine whether a subject should be subjected to a more invasive diagnostic method, such as PET. For example, in some embodiments, a subject whose tau phosphosite data indicates presence of a tauopathy such as AD or progression to a more advanced stage of tauopathy such as AD may be selected to receive further diagnostic tests, such as tau-based PET or amyloid beta-based PET, for example, in order to further confirm the presence of disease or a particular disease stage. In some embodiments, tau phosphosite data may be used to determine whether a subject should receive a cognitive test, for example, in order to further confirm presence of disease or a particular disease stage.

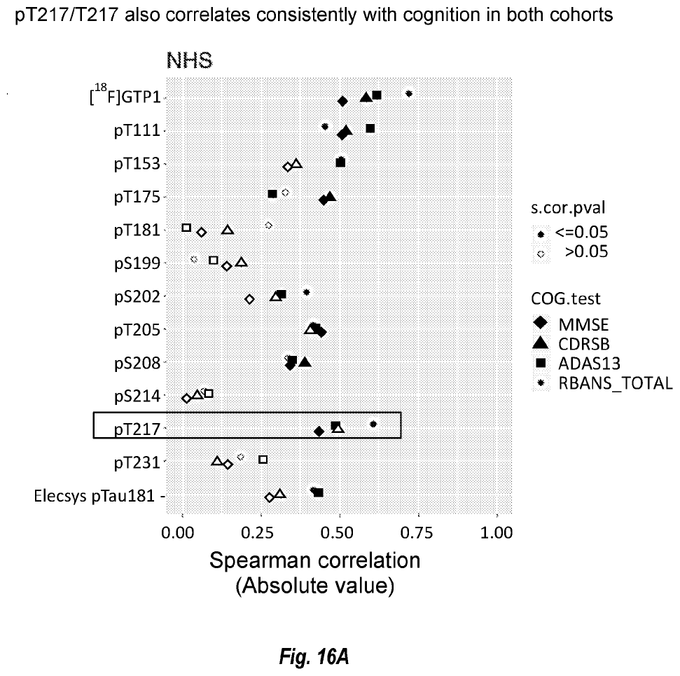

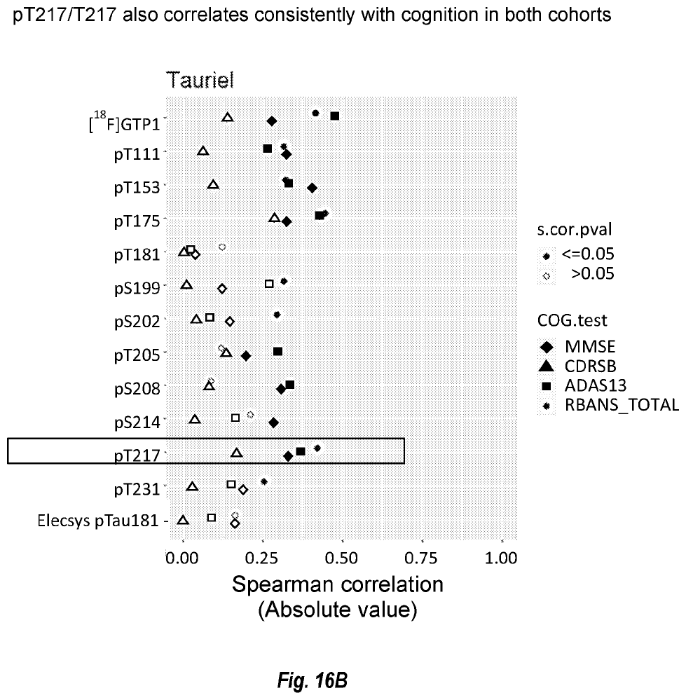

In some embodiments, the above methods may also be used to prognose cognitive status or progression. Thus, in some embodiments, the disclosure relates to the above methods for use in predicting cognitive status or predicting cognitive progression in a human subject with a tauopathy such as AD. In some embodiments, the methods comprise detecting the level of one or more of pT111, pT153, pT175, pS202, pT205, pS208, and pT217 in a biological sample from the subject (e.g. a CSF sample). (See, e.g., FIGS. 16A-B.) In some embodiments, the method comprises detecting the level of one or more of pT111, pT153, pT175, and pT217 in a biological sample from the subject (e.g. a CSF sample). In some embodiments, the method comprises detecting the level of one or more of pT153, pT175, and pT217 in a biological sample from the subject (e.g. a CSF sample). In some embodiments, the method comprises detecting the level of pT153 and/or pT175 plus pT217 in a biological sample from the subject (e.g. a CSF sample).

The disclosure herein also relates to methods of predicting likelihood that a human subject with a tauopathy such as AD will be responsive to an anti-tau antibody. Thus, in some aspects, methods described above may be used to predict likelihood of response of a subject to an anti-tau antibody. In further embodiments, the disclosure relates to methods of treating a subject with a tauopathy with an anti-tau antibody, wherein, prior to administration of the antibody, the subject has been tested according to one of the above methods. In some embodiments, the tauopathy is AD. In some such cases, the method is used to determine that the subject receiving treatment with the anti-tau antibody has a level of tested tau phosphosites corresponding to a level found in subjects showing clinical signs of AD, such as a level found in prodromal, mild, or moderate AD subjects. Thus, the method comprises using results from a method above to determine that a subject has a level of tested tau phosphosites corresponding to the level found in prodromal, mild, or moderate AD subjects, and then treating the subject with the anti-tau antibody. In some embodiments, the subject is re-tested for the tau phosphosites following administration of the anti-tau antibody. In some embodiments, an anti-tau antibody comprises, for example, gosuranemab (also known as BIIB092), ABBV-8E12, zagotenemab (also known as LY3303560), or semorinemab (also known as RO7105705, Mtau9937A, or RG6100). In some embodiments, the anti-tau antibody comprises semorinemab. In some embodiments, the subject is tested for levels or ratios of the tau phosphosites described herein before, during, and/or after administration of an anti-tau gene therapy, such as an AAV-tau therapeutic or a tau anti-sense oligonucleotide therapeutic.

Accordingly, in further embodiments, a method as described above may be used, for example, before and after treatment with an anti-tau antibody, or before and during treatment with an anti-tau antibody to determine likelihood of response of the subject to the antibody. For example, the method may be performed prior to start of treatment with the antibody and may be performed again at some point after treatment has begun. In some embodiments, an anti-tau antibody comprises, for example, gosuranemab (also known as BIIB092), ABBV-8E12, zagotenemab (also known as LY3303560), or semorinemab (also known as RO7105705, Mtau9937A, or RG6100). In some embodiments, the anti-tau antibody comprises semorinemab.

In any of the methods herein, the level of a tau phosphosite may be measured as the level of a peptide comprising the phosphorylated amino acid residue (e.g. pT153), or as the ratio of the level of a peptide comprising the phosphorylated amino acid residue over the total level of peptide(s) comprising the phosphorylated and unphosphorylated amino acid residue (e.g. pT153/T153).

In any of the methods herein, the level of a tau phosphosite may be detected by liquid chromatography (LC) followed by mass spectrometry (MS), such as tandem mass spectrometry (MS/MS). In such methods, the level of a peptide comprising a particular tau phosphosite (e.g. pT175 or pT175/T175) may be determined from a mass spectrometry analysis (e.g., from the peak height or integration of the peak corresponding to a particular site). In any of the method herein, determining the level of the tau phosphosite may comprise determining the level of the phosphorylated tau site (e.g., from the height or integration of the peak in a MS analysis corresponding to the appropriate phosphorylated peptide). In other embodiments, determining the level of the tau phosphosite may comprise determining the ratio of the level of the phosphorylated site (e.g., determined from a MS analysis as above) to the level of the unphosphorylated site (e.g., from the height or integration of the peak in a MS analysis corresponding to the appropriate unphosphorylated peptide). An exemplary such ratio is expressed, for example, as pT217/T217 or pS202/S202. Alternatively, in yet other cases, determining the level of the tau phosphosite may comprise determining the ratio of the level of the phosphorylated site to the level of the site as a whole, phosphorylated plus unphosphorylated. This would be expressed, for example, as, e.g., pT217/total T217. Thus, the “level” as recited in this context can actually be an amount as well as a ratio of phosphorylated levels to unphosphorylated levels or of phosphorylated levels to total levels.

In any of the methods herein, the tau phosphosite is assayed in a biological sample from the subject. The biological sample, in some aspects, is a cerebrospinal fluid (CSF) sample. In other aspects, the sample can be a brain interstitial fluid (ISF) sample.

As described herein, a tau PET or tau-based PET (positron emission tomography) tracer refers to a PET tracer used to track deposits of tau protein in the brain. Examples of tau PET tracers include, for example, ¹⁸F GTP1, ¹⁸F FDDNP, ¹⁸F THK5317, ¹⁸F THK5351, ¹⁸F T807, ¹¹C PBB3, ¹⁸F MK-6240, ¹⁸F RO-948, ¹⁸F PI-2620, ¹⁸F PM-PBB3, ¹⁸F JNJ311, and ¹⁸F JNJ067, among others. In some aspects, a tau PET tracer is 18F GTP1. ¹⁸F GTP1 is a tau PET tracer that selectively binds to pathological tau in Alzheimer's disease and has demonstrated high selectivity for tau pathologies over amyloid-beta pathologies in prior studies. (See Sanabria Bohorquez et al., 2019, refs. 36-37.) In some embodiments, an amyloid-beta PET tracer is used, such as ¹⁸F-florbetapir.

In the methods herein, the subject is a human subject unless expressly stated otherwise.

In the methods herein, a subject may have or be suspected to have a tauopathy. In some aspects, the subject has or is suspected to have Alzheimer's disease (AD). In some aspects, the subject has or is suspected to have a tauopathy such as, amyotrophic lateral sclerosis, Parkinson's disease, Creutzfeldt-Jacob disease, Dementia pugilistica, Down's Syndrome, Gerstmann-Straussler-Scheinker disease, inclusion-body myositis, prion protein cerebral amyloid angiopathy, traumatic brain injury, amyotrophic lateral sclerosis/parkinsonism-dementia complex of Guam, Non-Guamanian motor neuron disease with neurofibrillary tangles, argyrophilic grain dementia, corticobasal degeneration, diffuse neurofibrillary tangles with calcification, frontotetemporal dementia, frontotemporal dementia with parkinsonism linked to chromosome 17, Hallevorden-Spatz disease, multiple system atrophy, Niemann-Pick disease type C, Pallido-ponto-nigral degeneration, Pick's disease, progressive subcortical gliosis, progressive supranuclear palsy, Subacute sclerosing panencephalitis, Tangle only dementia (or tangle predominant dementia), Postencephalitic Parkinsonism, Myotonic dystrophy, PART (primary age-related Tauopathy), tangle predominant dementia, subacute sclerosis panencephalopathy, chronic traumatic encephalopathy (CTE), white matter tauopathy with globular glial inclusions, Lewy body dementia (LBD), mild cognitive impairment (MCI), glaucoma, familial British dementia, familiar Danish dementia, Guadeloupean Parkinsonism, neurodegeneration with brain iron accumulation, SLC9A6-related mental retardation, multiple sclerosis, HIV-related dementia, senile cardiac amyloidosis, or Huntington's disease.

As used herein, “prodromal” AD refers to an early stage AD in which the subject is functionally independent but has a deteriorating memory. In some embodiments, a “prodromal” AD subject may correspond to a Stage 2 subject in a 7-stage scale of AD (in which stages comprise (1) outwardly normal, (2) very mild/prodromal, (3) mild, (4) moderate, (5) moderately severe, (6) severe, and (7) very severe). As used herein, “mild” AD or an AD subject with “mild” decline refers to a subject experiencing memory decline such as forgetting something just read, remembering people's names, difficulty planning or organizing, and repeatedly asking the same question. In some embodiments, a “mild” AD subject may correspond to a Stage 3 subject in a 7-stage scale of AD. As used herein, “moderate” AD refers to a subject experiencing more pronounced decline, such as forgetting details about himself, forgetting what month or season it is, having trouble with tasks such as cooking or ordering from a menu. In some embodiments, a “moderate” AD subject may correspond to a Stage 4 subject in a 7-stage scale of AD. In contrast, a “cognitively normal” (CN) subject determined to have no detectable impairment in memory or symptoms of dementia, i.e., outwardly normal.

Certain exemplary embodiments of this disclosure include the following, as well as those provided in the claims below:
1. A method for determining whether a human subject is likely to have AD (such as prodromal, mild, or moderate AD) by detecting the level of at least one tau phosphosite in a CSF sample from the subject (for example by liquid chromatography mass spectrometry (e.g. tandem mass spectrometry) analysis (LC-MS or LC-MS/MS)).
2. The method of (1), wherein the subject:

a. Is known to be Aβ+, for example, based on an amyloid beta PET scan;

b. Has not received an amyloid beta PET scan;

c. Has not received a tau PET scan, such as a tau ([¹⁸F]GTP1) PET imaging scan; or

d. Has not received either an amyloid beta PET scan or a tau PET scan.
3. The method of (1) or (2), for determining whether the human subject is likely to have prodromal AD.
4. The method of (1) or (2), for determining whether the human subject is likely to have mild AD.
5. The method of (1) or (2), for determining whether the human subject is likely to have moderate AD.
6. A method of staging an AD or potential AD subject, comprising detecting the level of at least one tau phosphosite in a CSF sample from the subject (for example by liquid chromatography mass spectrometry (e.g. tandem mass spectrometry) analysis (LC-MS or LC-MS/MS)), and determining whether the subject is likely to have prodromal AD, mild AD, or moderate AD based on the level of the at least one tau phosphosite in the sample.
7. The method of any one of (1) to (6), further comprising comparing the phosphosite level(s) to the level(s) previously observed in CN subjects (such as Aβ+ but CN subjects, Aβ− CN subjects, or all CN subjects regardless of Aβ status).
8. The method of (6) or (7), wherein levels of at least two of phosphosites are detected.
9. A method of staging an AD or potential AD subject as likely to be CN or to have prodromal, mild, or moderate AD, comprising detecting the level of at least one tau phosphosite in a CSF sample from the subject (for example by liquid chromatography mass spectrometry (e.g. tandem mass spectrometry) analysis (LC-MS or LC-MS/MS)), and determining whether the subject is likely to have prodromal AD, mild AD, or moderate AD based on the level of the at least one tau phosphosite in the sample compared to levels of the phosphosite in control samples of prodromal AD, mild AD, moderate AD, and/or CN subjects, wherein levels of at least one of pT111, pT153, pT175, pS199, pS202, pT205, pS208, pS214, pT217, and pT231 are detected, and wherein levels of pT175 and pS202 decrease with increasing AD stage, levels of pT111, pT153, pS199, pS208, pS214, pT217 and pT231 are higher in AD subjects than in CN subjects, and levels of pS214 and pT231 are higher in prodromal AD subjects than in CN subjects but lower in mild and moderate AD subjects than in prodromal AD subjects.
10. The method of any one of (1) to (9), comprising detecting the level of one or more of the following tau phosphosites in the CSF sample: pT111, pT153, pT205, pS208, pS199, pT217, pT181, pS214, pT231, pT175, and pS202.
11. The method of any one of (1) to (10), wherein the method comprises detecting the level of one or more of pT111, pT153, pT205, pS208, pS199, and pT217, and optionally, determining whether the level of the one or more phosphosites is elevated in comparison to CN subjects (such as Aβ+ but CN subjects, Aβ− CN subjects, or all CN subjects regardless of Aβ status).
12. The method of any one of (1) to (11), comprising detecting the level of one or more of pT111, pT153, or pT175.
13. The method of (12), wherein the method comprises determining whether the level of pT111 and/or pT153 is elevated in comparison to CN subjects (such as Aβ+ but CN subjects, Aβ− CN subjects, or all CN subjects regardless of Aβ status); and/or wherein the method comprises determining whether the level of pT175 is reduced in comparison to CN subjects (such as Aβ+ but CN subjects, Aβ− CN subjects, or all CN subjects regardless of Aβ status) and/or in comparison to prodromal or mild AD subjects.
14. The method of any one of (1) to (13), comprising detecting the level of two or more of pT111, pT153, pT205, pS208, pS199, and pT217.
15. The method of any one of (1) to (14), wherein the method comprises detecting the level of one or more of pT181, pS214, and pT231, and optionally, determining whether the level of the one or more phosphosites is elevated in comparison to CN subjects (such as Aβ+ but CN subjects, Aβ− CN subjects, or all CN subjects regardless of Aβ status), or is elevated in comparison to mild or moderate AD subjects.
16. The method of (15), comprising detecting the level of two or more of pT181, pS214, and pT231, or all three of pT181, pS214, and pT231.
17. The method of (15), wherein the method comprises detecting the level of pS214, and optionally, determining whether the level of pS214 is elevated in comparison to CN subjects (such as Aβ+ but CN subjects, Aβ− CN subjects, or all CN subjects regardless of Aβ status), or is elevated in comparison to mild or moderate AD subjects.
18. The method of any one of (1) to (17), wherein the method comprises detecting the level of one or both of pT175 and pS202, and optionally, determining whether the level of the one or more phosphosites is reduced in comparison to CN subjects (such as Aβ+ but CN subjects, Aβ− CN subjects, or all CN subjects regardless of Aβ status) and/or whether the level is elevated in comparison to mild or moderate AD subjects.
19. The method of any one of (1) to (18), wherein the method comprises detecting the level of no more than 11 phosphosites.
20. The method of (19), wherein the method comprises detecting the level of no more than 8 phosphosites.
21. The method of (19), wherein the method comprises detecting the level of no more than 6 phosphosites.
22. The method of (19), wherein the method comprises detecting the level of no more than 4 phosphosites.
23. The method of (19), wherein the method comprises detecting the level of one, two, or all of pT111, pT153, and pT175, and also at least one of pT181, pS199, pS202, pT205, pS208, pS214, pT217, and pT231.
24. The method of (19), wherein the method comprises detecting the level of pT217, pT153, pT181, pT111, pS208, and pT231.
25. The method of any one of (1) to (24), wherein the method comprises detecting the level of (a) one or more of pT111, pT153, pT205, pS208, pS199, and pT217; (b) one or more of pT181, pS214, and pT231; and/or (c) one or both of pT175 and pS202.
26. The method of any one of (1) to (24), wherein the method comprises detecting the level of (a) one or more of pT111, pT153, pT205, pS208, pS199, and pT217; (b) one or more of pT181, pS214, and pT231; and (c) one or both of pT175 and pS202.
27. The method of any one of (1) to (26), wherein the level of the phosphosite(s) is calculated as the ratio of the level of the phosphorylated site to the level of its corresponding unposphorylated site (e.g., by calculating the ratio of the level of a peptide comprising the phosphorylated site to the level of the corresponding unphosphorylated peptide in a MS analysis).
28. The method of any one of (1) to (26), wherein the method is used in place of tau PET and/or amyloid beta PET.
29. The method of any one of (1) to (26), wherein the subject has not had tau PET and/or amyloid beta PET and wherein the method is used to determine whether the subject should receive tau PET and/or amyloid beta PET.
30. The method of any one of (1) to (29), wherein the method is used to determine whether the subject should receive an anti-tau antibody therapy.
31. A method of treating a subject with an anti-tau antibody, wherein the subject has been tested prior to treatment according to the method of any one of (1) to (29) and found to have a level of tau phosphosite(s) corresponding to that of an AD subject (such as a prodromal, mild, or moderate AD subject).
32. A method of treating a subject with an anti-tau antibody, wherein the subject has been tested prior to treatment according to the method of any one of (1) to (29), the method comprising determining that the subject has a level of tau phosphosite(s) corresponding to that of an AD subject (such as a prodromal, mild, or moderate AD subject), and subsequently administering an anti-tau antibody to the subject.
33. The method of (31) or (32), wherein the subject is re-tested according to the method of any one of (1) to (29) following administration of the anti-tau antibody.
34. The method of any one of (30) to (33), wherein the anti-tau antibody is gosuranemab, ABBV-8E12, zagotenemab, or semorinemab.
35. The method of (34), wherein the anti-tau antibody is semorinemab.
36. The method of any one of (1) to (29), wherein the method is used to prognose cognitive status or AD stage (e.g., prodromal, mild, moderate) if the subject, either alone or in combination with other assays such as tau PET, amyloid PET, MRI, Elecsys® pTau181, and cognitive function assays.

### Examples

### I. Measuring Tau in CSF

Tau PET is believed to be reflective of accumulation of tau protein deposits in the brain of a subject. [¹⁸F]GTP1 is a PET tracer in development that binds to tau pathology and enables the study of tau propagation in AD. Other tau PET tracers are described herein. PET tracer development may take a significant amount of time and can be a costly investment.

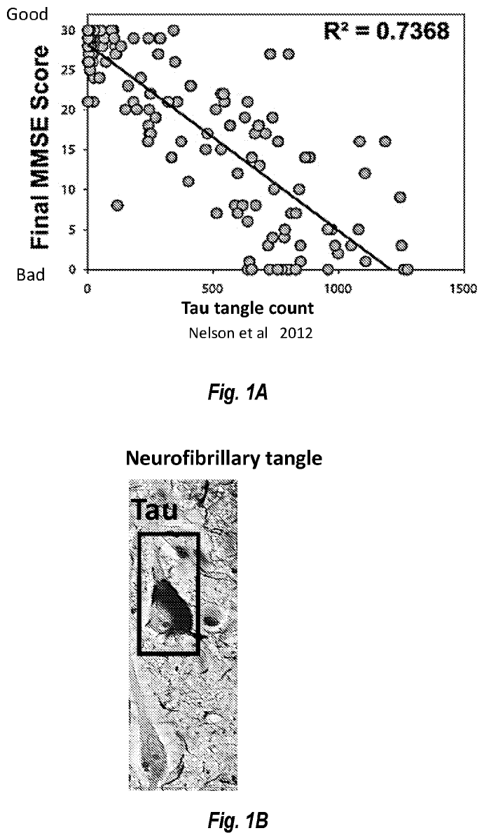

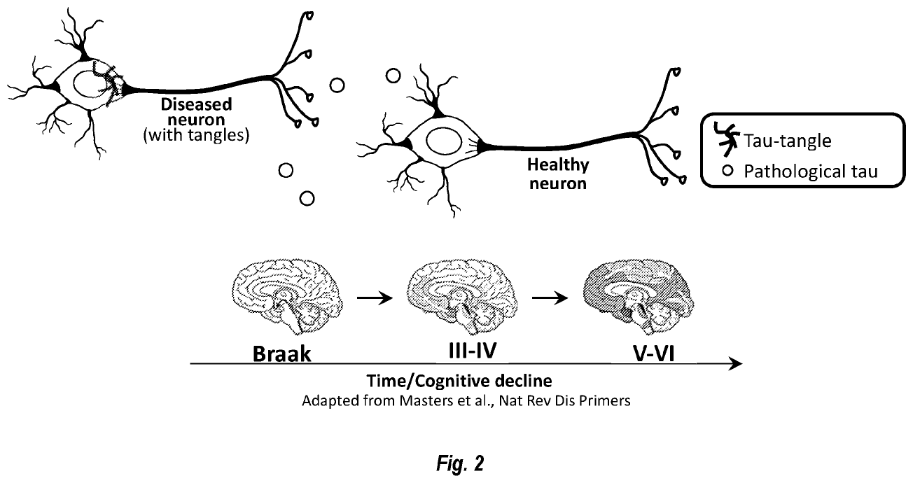

Tau found in cerebrospinal fluid (CSF) or other bodily fluids such as brain interstitial fluid (ISF) may be reflective of a circulating, soluble pool of tau protein. It is hypothesized, for example, that as tau aggregates (accumulation of tau over decades), CSF tau increases, and there is cognitive decline. FIG. 1A shows how tau pathology correlates with cognition and spreads with increasing disease severity (MMSE scores and Tau tangle count). FIG. 1B shows tau present in a neurofibrillary tangle. Hyperphosphorylated tau is one of the major components of tangles in Alzheimer's disease. The tau protein can be phosphorylated at >80 sites. FIG. 2 shows a depiction of how tau pathology may spread in a prion-like manner.

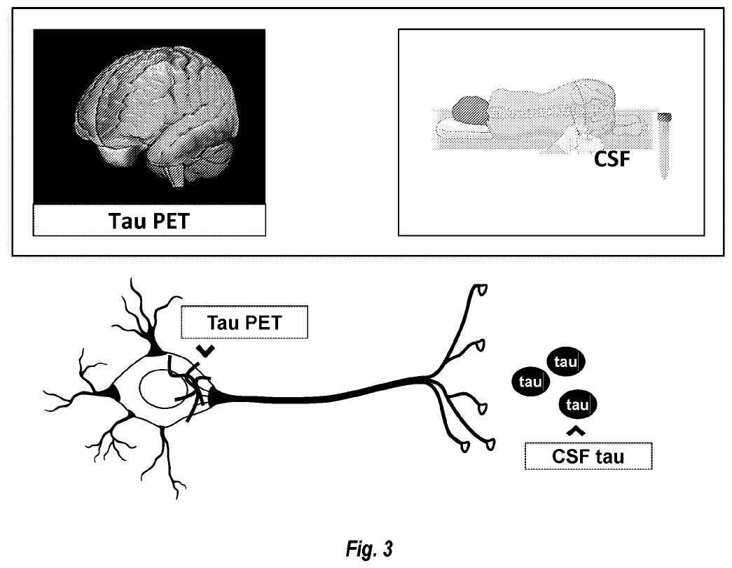

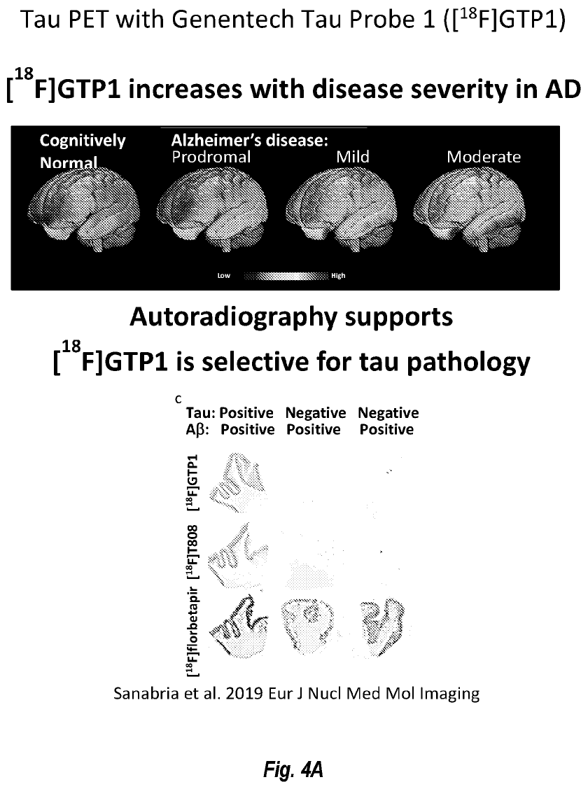

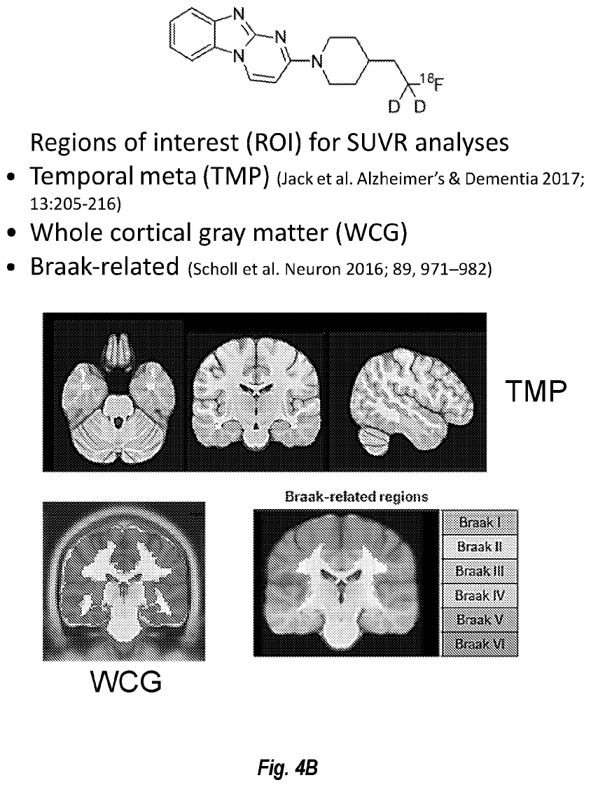

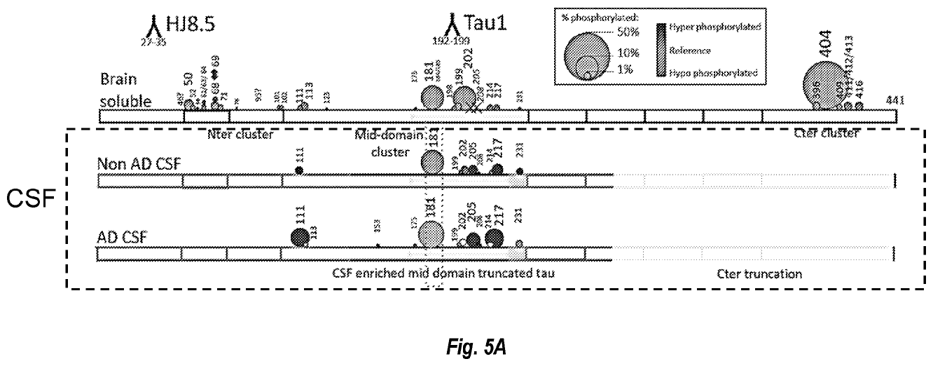

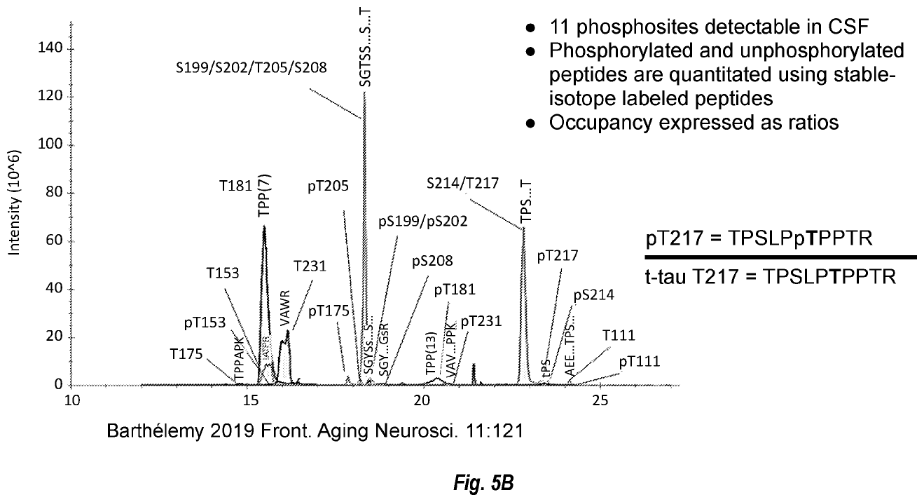

The tau protein can be phosphorylated at >80 sites. Some studies have assessed changes in phosphorylated tau protein in the CSF by measuring phosphorylation at threonine 181 (pT181). Others have shown that pT181 in CSF is highly correlated with the total level of CSF tau (“total tau” or “t-tau”). FIG. 3 shows a depiction of ways to measure tau using positron emission tomography (PET) and cerebral spinal fluid (CSF)-based methods. FIGS. 4A-B show data from tau PET assays using a [¹⁸F]GTP1 (Genentech Tau Probe 1) PET tracer. FIG. 4A shows how [¹⁸F]GTP1 increases with disease severity in AD, and is selective for tau pathology. FIG. 4B shows regions of interest for tau PET SUVR analyses in temporal meta (TMP) and whole cortical grey matter (WCG) and Braak-related regions. FIG. 5A shows phosphorylation sites (or phosphosites) across the domains of the tau protein and percent (%) phosphorylation at those sites (red=hyperphosphorylated (e.g., at 111, 205, and 217 in both Non AD and AD CSF); green=hypophosphorylated (e.g., at 202 in Non AD CSF) in brain soluble CSF, non-AD CSF, and AD CSF measured by multiplexed IP-LC/MS. CSF Tau was also measured by an Elecsys® pTau181 assay (mid-domain area shaded in purple). We are interested in studying how tau PET and CSF tau levels change in AD and how they are related.

An ELISA method has been used for measurement of t-tau in CSF, which is based on three monoclonal antibodies binding to the mid region of the protein (39), meaning that the sum of full length tau and all fragments containing the mid region is monitored. However, this ELISA assay is not able to distinguish between full-length tau or mid-domain fragments, and either N-terminal or C-terminal tau fragments.

II. ¹⁸F-GTP1 PET Studies—Natural History Study (NHS) and e0048 Study

GN30009 (NCT02640092) is a longitudinal study designed to evaluate the natural history of tau pathology (or referred to herein as the “Natural History Study or NHS”) in individuals with prodromal Alzheimer's disease, in individuals with mild or moderate Alzheimer's disease dementia, and in healthy volunteers.

### Participants

Baseline CSF indices and ¹⁸F-GTP1 PET scans were obtained from a subset of research participants, as previously described in Sanabria Bohorquez et al. (2019) (36, 37). (See Table 1.)

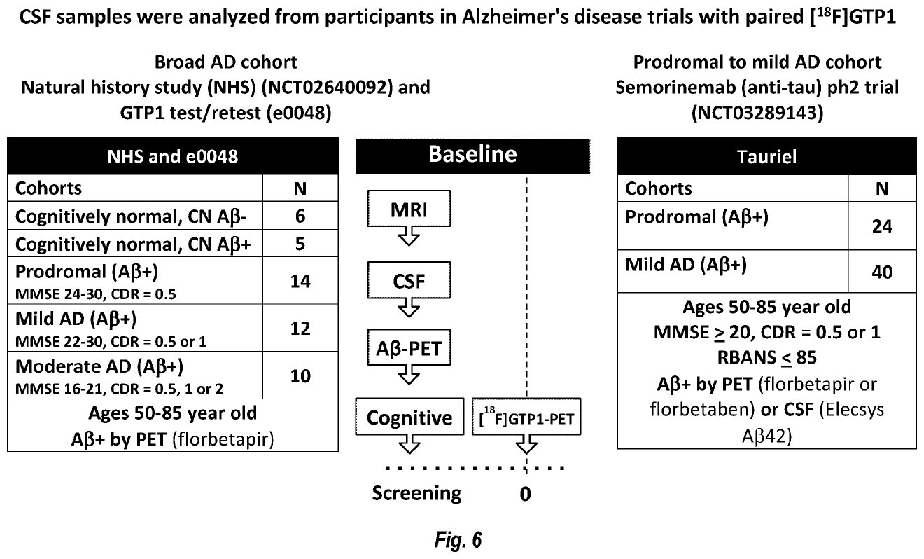

The participants in this study received anatomical MRI, amyloid PET for inclusion, and received 4 GTP1 scans over 18 months. Participants underwent cognitive evaluation at each tau PET time point, as well optional CSF collection at baseline and 12 months. There were 4 cohorts in the study: CN amyloid positive and negative, prodromal AD, mild AD, and moderate AD. All AD participants had a positive amyloid scan. (See FIG. 6, left panel, referring to the “NHS study”.) For GN30009, all participants in the Alzheimer's disease subgroups were required to have a screening 18F-florbetapir PET scan that was deemed amyloid-b positive by visual read by two central raters and a screening brain MRI scan that was consistent with a diagnosis of Alzheimer's disease and did not show significant evidence of non-Alzheimer's disease neurological disease that might contribute to cognitive impairment.

TABLE 1
Demographic Data in Three Clinical Cohorts - GN30009

| | Age, years | Gender | p-tau, ng/L | | p-tau, ng/L | | Aβ, ng/L | |
|---|---|---|---|---|---|---|---|---|
| | Mean (range) | Male/Female | Mean (range) | | Mean (range) | | Mean (range) | |
| Pilot study | | | | | | | | |
| AD (n = 21) | 75 (59-85) | 9/12 | 92 | (71-195) | 735 | (520-1370) | 362 | (220-460) |
| Control (n = 20) | 63 (42-84) | 14/6 | 40 | (26-53) | 217 | (120-260) | 806 | (670-1050) |
| Clinical study | | | | | | | | |
| AD (n = 37) | 73 (53-84) | 13/24 | 82 | (50-136) | 728 | (304-1396) | 372 | (191-533) |
| Control (n = 45) | 73 (60-83) | 15/30 | 46 | (19-74) | 330 | (88-564) | 913 | (217-1698) |
| GTP1 study | | | | | | | | |
| Control (n = 11) | 64 (51-71) | 6/5 | 20 | (11-36) | 386 | (165-660) | 908 | (542-1363) |
| Prodromal AD (n = 15) | 70 (56-83) | 8/7 | 32 | (11-72) | 619 | (204-1332) | 685 | (392-1102) |
| Mild AD (n = 13) | 70 (59-80) | 5/8 | 37 | (16-86) | 699 | (327-1558) | 720 | (393-1032) |
| Moderate AD (n = 10) | 72 (68-76) | 8/2 | 37 | (20-72) | 719 | (379-1392) | 522 | (309-931) |

Biomarker data are presented in ng/l. AD = Alzheimer's disease; p-tau = tau phosphorylated at 181. INNOTEST ELISA assays were used to analyze amyloid-β₁₋₄₂, t-tau, and p-tau. With the exception of amyloid-β₁₋₄₂ and p-tau in the GTP1 study, which was analyzed using Elecsys ® assays.

Baseline assessments for these studies included MRI, PET imaging with 18F-GTP1 and Aβ PET tracers, cognitive screening, and collection of CSF for analysis.

### PET Imaging

The imaging assessments for both studies were performed at the same imaging center. PET images were acquired on a Siemens HR+ or Siemens Biograph™ 6 PET-CT scanners. Images were corrected for attenuation, random coincidences, scatter, and decay. Images were reconstructed using harmonization parameters derived from Hoffman 3-D Brain Phantom™ studies using 18F in each scanner to insure the same image resolution in all images.

### ¹⁸F-GTP1 PET Imaging

¹⁸F-GTP1 scans were performed following procedures previously described (36, 37). In brief, ¹⁸F-GTP1 images were acquired over a 30-min window starting 60 min postinjection after a mean (SD) bolus injection of 343 (31) MBq. 18F-GTP1 standardized uptake value ratios (SUVRs) were calculated using the cerebellar gray as reference. Data were reported for an Alzheimer's disease temporal meta-region of interest (38).

### MRI

MRI was performed to determine participant eligibility, for coregistration with 18F-GTP1 and 18F-florbetapir PET images, and for volumetric analyses. The 3D sagittal T1-weighted MPRAGE sequences were used for volumetric analyses. Images were collected with 1 mm² in-plane resolution, 1.0-1.2 mm slice thickness, 256 mm-256 mm matrix, and a 240 mm field of view.

e0048 Study

A second study, e0048, was designed to evaluate the test/retest reliability of 18F-GTP1 binding parameters in individuals with Alzheimer's disease dementia and healthy volunteers. (See FIG. 6, left panel, referring to the “e0048 study”.)

III. CSF Phosphotau217 Correlates with [¹⁸F]GTP1 Pet in Alzheimer's Disease: A Multiple Phosphosite Analysis of CSF Tau Species

Objective: The objective of this study was to explore which phosphosites on tau in CSF samples correlate with [¹⁸F]GTP1 PET in Alzheimer's disease (AD) using an expanded phospho-tau LC-MS/MS assay. Neurofibrillary tangles can be visualized in Alzheimer's patients with Tau PET tracers like [¹⁸F]GTP1. CSF pTau181 as determined in an Elecsys® pTau181 assay, which is diagnostic for AD, is modestly correlated with Tau PET. We identified other phosphosites that may have different or stronger correlations with [¹⁸F]GTP1 PET.

Methods: Paired CSF and [¹⁸F]GTP1 PET data were collected in clinical studies (the NCT02640092 “NHS Study” combined with the “e0048 study,” and the NCT03289143 “Tauriel Study”) from cognitively normal subjects (11) and prodromal (n=37), mild (n=52), and moderate (n=10) AD patients. (See FIG. 6 for summary.) Tau PET uptake was measured as SUVR in a temporal meta-ROI (cerebellar gray reference region). CSF was used for Elecsys® pTau181 and Elecsys® t-tau assays and for a multiplexed nano-LC-MS/MS assay that quantified phosphorylated and unphosphorylated peptides at 11 phosphosites. (See FIG. 5B.) Relationships between tau phosphorylation levels at each site (measured as ratios of phosphorylated peptide levels to total peptide levels) and [18F]GTP1 SUVR were assessed by Spearman correlations.

FIG. 5B shows that phosphorylation at multiple sites on the tau protein may be detected by LC-MS. CSF Tau was measured by multiplexed IP-LC/MS (See 40: Barthélemy 2019 Front. Aging Neurosci. 11:121). Eleven phosphosites were detectable in CSF samples. Phosphorylated and unphosphorylated peptides were quantified using stable-isotope labeled peptides in the multiplexed IP-LC/MS assay. Phosphosite level (occupancy) was measured as ratio of phosphorylated peptide to unphosphorylated peptide (i.e., both phosphorylated and unphosphorylated). Thus, for example, for pT217:

pT217=TPSLPpTPPTR

T217=TPSLPTPPTR)

Occupancy=pT217/T217=(TPSLPpTPPTR)/(TPSLPTPPTR)

Comparison of GTP1 PET Studies and Semorinemab (Tauriel) Study: FIG. 6 shows analysis of CSF samples from participants in Alzheimer's disease trials with paired [¹⁸F]GTP1. The utility of these different CSF phosphorylated species was evaluated by comparing correlation of the species with AD pathology as measured with imaging (Florbetapir (FBP) PET, ¹⁸F GTP1 PET and MRI) and cognition in the study cohorts from the 18 month trials.

A “broad AD cohort” or “NHS/e0048 cohort” (a combination of the Natural History study (NHS, Clinical Trial No. NCT02640092) and GTP1 test/retest(e0048) study cohorts) included cognitive normals (CN) through to moderate AD subjects. (FIG. 6, left panel.) All subjects were screened with florbetapir PET for amyloid beta.

A “semorinemab cohort” or “Tauriel cohort” included subjects from a separate phase 2 trial (NCT03289143) for semorinemab (an anti-Tau antibody) (the “Tauriel study”). Prodromal and mild AD patients were evaluated using baseline samples. (FIG. 6, right panel.) The enrollment criteria, clinical diagnosis, and patient population were different compared to those of the Natural History study (NHS). The semorinemab trial added RBANs criteria, and patients had an option to be enrolled by A-beta by PET or CSF. The majority of the patients were enrolled with PET.

These two cohorts (broad cohort and semorinemab cohort) were compared and studied further.

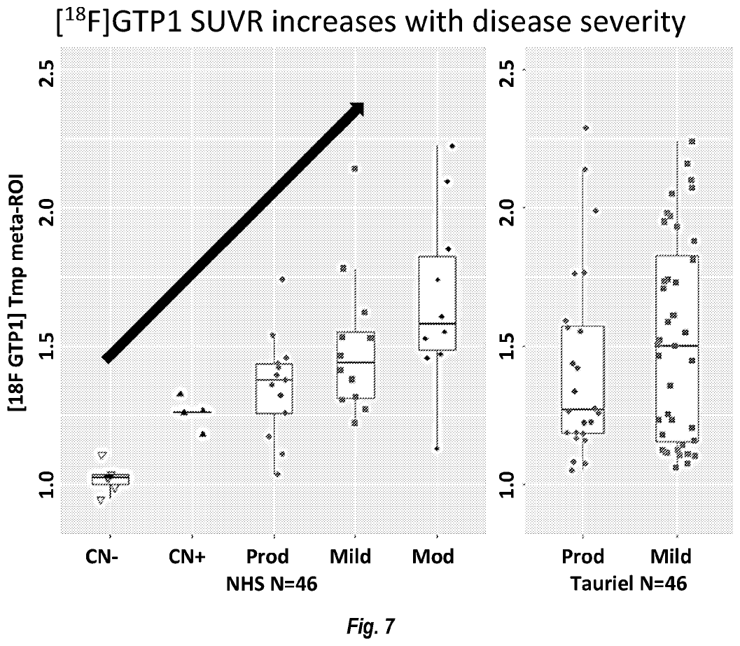

FIG. 7 shows that [¹⁸F]GTP1 SUVR increases with disease severity in the broad AD cohort (labeled NHS in the figure). While it didn't reach significance, the signal tended to be higher in mild AD than prodromal AD and higher in moderate AD compared to mild AD. A similar non-significant trend was seen in the Tauriel cohort.

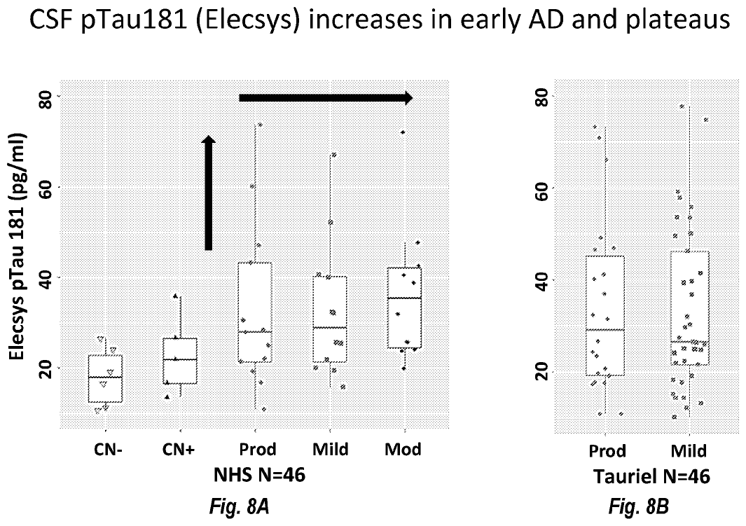

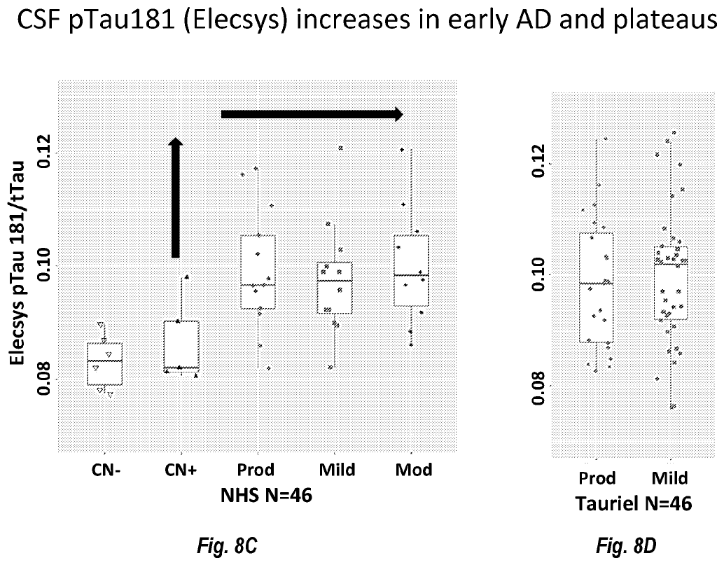

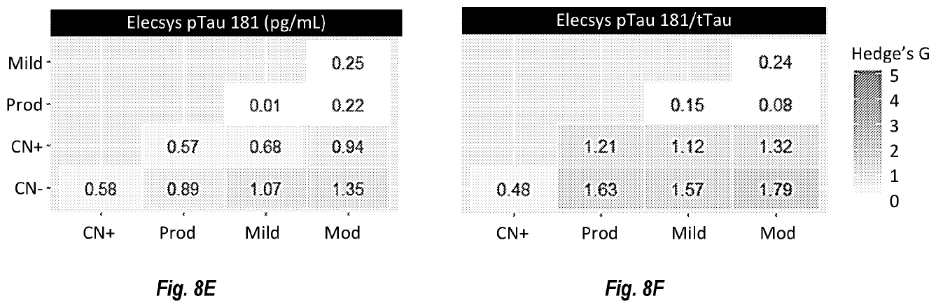

FIGS. 8A-F show that CSF pTau181 (measured using Elecsys®) increases in early AD, i.e. in prodromal AD, and then plateaus in later stages (i.e., mild to moderate AD). CSF pTau181 (a well validated tau diagnostic biomarker) was consistent with previous reports and increased in early (prodromal) AD and then plateaued.

To determine whether the phosphorylated to unphosphorylated occupancy ratio can be used in a MS assay to explore occupancy, we studied whether the ratio of Elecsys® pTau 181 to Elecsys® t-tau performed any differently compared to the level of pTau181 determined by Elecsys® pTau181 assay alone. Visually and numerically, based on the effect size, the Elecsys® pTau181 to t-tau ratio provided a greater separation between the cohorts, as shown in the two tables below each graph in the figure.

IV. Comparison of 11 CSF P-Tau Sites Highlights Stronger Association of T217 Phosphorylation with Amyloid and Tau Pathologies as Measured by PET in Alzheimer's Disease

Objective: We used a mass spectrometry (MS)-based method to evaluate the relationship between disease-associated phosphorylation sites in CSF and pathology as measured by tau PET ([¹⁸F]GTP1) and amyloid PET ([¹⁸F]florbetapir or [¹⁸F]florbetaben) in Alzheimer's disease (AD) patients.

Methods: We compared the changes in tau phosphorylation ratios to brain imaging ([¹⁸F]florbetapir, [¹⁸F]GTP1 PET, and MRI), and cognition across clinical stages of AD in two different cohorts A) a full clinical range AD cohort including cognitively normal-amyloid negative (n=6) and -positive (n=5), prodromal (n=13), mild (n=12), and moderate AD patients (n=10); and B) an early AD cohort including prodromal (n=24) and mild (n=40) AD patients.

Results: Cross-sectional analyses suggest different profiles of phosphorylation ratios at different sites with disease progression. Among the 11 phosphorylated tau sites investigated, pT217 had the strongest association with SUVR measures from [¹⁸F]GTP1 and amyloid PET, and most consistent correlation with cognition across cohorts.

Conclusions: Our results support site-specific trajectories of tau phosphorylation changes in CSF occurring concomitantly with brain AD tau pathology measured by PET. These novel fluid biomarkers for tau may aid in diagnosis and management of AD.

1. Results

1.1 Demographics: Participant demographics and group means for each of the biomarkers measured are shown in Table 2 (below). Mean age (˜70 y) was similar between cohorts, with the exception of the amyloid-negative cognitive normal group (59 y). The prodromal to mild patients in the early AD cohort on average had poorer performance on cognitive assessments, in comparison with prodromal to mild patients in the full AD cohort.

TABLE 2
Patient demographic and disease characteristics expressed as mean (SD).

| | Full Clinical Range | | | | | Early AD | |
|---|---|---|---|---|---|---|---|
| | CN− | CN+ | Prodromal | Mild | Moderate | Prodromal | Mild |
| | (n = 6) | (n = 5) | (n = 15) | (n = 13) | (n = 10) | (n = 24) | (n = 40) |
| Age | 59 | 68.63 | 69.81 | 70.6 | 70.87 | 70.71 | 69.28 |
| | (8.22) | (3.38) | (6.85) | (6.14) | (7.02) | (7.06) | (7.47) |
| Female | 3 | 2 | 7 | 8 | 2 | n = 10 | n = 17 |
| | (34%) | (40%) | (47%) | (13%) | (20%) | | |
| MMSE | 29.33 | 29.00 | 28.31 | 26.58 | 17.60 | 25.83 | 23.03 |
| | (0.82) | (0.71) | (1.18) | (2.47) | (2.76) | (2.70) | (2.48) |
| CDRSB | 0.00 | 0.10 | 1.58 | 3.18 | 6.22 | 2.56 | 4.72 |
| | (0.00) | (0.22) | (0.91) | (1.27) | (1.72) | (1.15) | (1.85) |
| ADAS13 | 12.34 | 8.00 | 15.31 | 19.24 | 39.78 | 22.53 | 29.57 |
| | (4.72) | (4.75) | (6.11) | (6.87) | (7.29) | (7.39) | (6.84) |
| RBANS-Total | 98.50 | 91.8 | 85.23 | 77 | 60.5 | 76.13 | 61.24 |
| | (3.54) | (11.56) | (9.28) | (14.55) | (12.12) | (13.50) | (11.83) |
| [¹⁸F]GTP1-PET | 1.02 | 1.26 | 1.36 | 1.5 | 1.67 | 1.42 | 1.52 |
| Temp ROI SUVR | (0.05) | (0.05) | (0.19) | (0.26) | (0.32) | (0.34) | (0.37) |
| [¹⁸F]GTP1-PET | 1.00 | 1.17 | 1.18 | 1.34 | 1.45 | n/a | n/a |
| WCG SUVR | (0.05) | (0.05) | (0.10) | (0.27) | (0.41) | | |
| [¹⁸F]GTP1-PET | 1.00 | 1.27 | 1.36 | 1.44 | 1.48 | n/a | n/a |
| Braak12 SUVR | (0.05) | (0.04) | (0.17) | (0.14) | (0.26) | | |
| [¹⁸F]GTP1-PET | 1.00 | 1.2 | 1.27 | 1.44 | 1.55 | n/a | n/a |
| Braak34 SUVR | (0.05) | (0.05) | (0.16) | (0.30) | (0.35) | | |
| [¹⁸F]GTP1-PET | 1.00 | 1.15 | 1.13 | 1.29 | 1.4 | n/a | n/a |
| Braak56 SUVR | (0.06) | (0.06) | (0.09) | (0.26) | (0.46) | | |
| Florbetapir-PET | 1.00 | 1.17 | 1.36 | 1.42 | 1.42 | 1.52 | 1.49 |
| SUVR | (0.06) | (0.09) | (0.15) | (0.11) | (0.13) | (0.24) | (0.21) |
| Elecsys ® pTau181 | 18.07 | 22.92 | 32.8 | 32.7 | 36.73 | 34.74 | 36.22 |
| (pg/ml) | (6.52) | (8.78) | (18.24) | (15.11) | (15.54) | (21.2) | (23.75) |
| Elecsys ® tTau | 217.02 | 261.54 | 317.28 | 326.48 | 359.21 | 331.33 | 354.1 |
| (pg/ml) | (77.36) | (82.36) | (140.77) | (118.16) | (117.92) | (168.76) | (199.17) |
| Elecsys ® | 0.083 | 0.086 | 0.099 | 0.098 | 0.1 | 0.1 | 0.098 |
| pTau181/tTau | (0.005) | (0.008) | (0.011) | (0.01) | (0.011) | (0.014) | (0.014) |
| pT111/T111 | 0.039 | 0.065 | 0.119 | 0.111 | 0.137 | 0.087 | 0.09 |
| | (0.015) | (0.028) | (0.044) | (0.034) | (0.020) | (0.035) | (0.035) |
| pT153/T153 | 0.002 | 0.003 | 0.009 | 0.009 | 0.009 | 0.007 | 0.007 |
| | (0.000) | (0.002) | (0.003) | (0.004) | (0.004) | (0.003) | (0.003) |
| pT175/T175 | 0.003 | 0.003 | 0.003 | 0.003 | 0.002 | 0.003 | 0.003 |
| | (0.001) | (0.001) | (0.001) | (0.000) | (0.001) | (0.001) | (0.001) |
| pT181/T181 | 0.237 | 0.300 | 0.347 | 0.308 | 0.28 | 0.329 | 0.317 |
| | (0.025) | (0.027) | (0.059) | (0.047) | (0.048) | (0.058) | (0.061) |
| pS199/S199 | 0.003 | 0.003 | 0.004 | 0.004 | 0.004 | 0.005 | 0.005 |
| | (0.001) | (0.001) | (0.001) | (0.002) | (0.001) | (0.002) | (0.001) |
| pS202/S202 | 0.039 | 0.045 | 0.039 | 0.039 | 0.034 | 0.04 | 0.037 |
| | (0.005) | (0.016) | (0.008) | (0.011) | (0.005) | (0.011) | (0.013) |
| pT205/T205 | 0.004 | 0.005 | 0.006 | 0.008 | 0.007 | 0.007 | 0.007 |
| | (0.001) | (0.002) | (0.002) | (0.002) | (0.001) | (0.002) | (0.002) |
| pS208/S208 | 0.000 | 0.001 | 0.001 | 0.001 | 0.001 | 0.001 | 0.001 |
| | (0.000) | (0.000) | (0.000) | (0.000) | (0.000) | (0) | (0) |
| pS214/S214 | 0.000 | 0.001 | 0.001 | 0.001 | 0.001 | 0.001 | 0.001 |
| | (0.000) | (0.000) | (0.000) | (0.000) | (0.000) | (0) | (0) |
| pT217/T217 | 0.015 | 0.026 | 0.061 | 0.063 | 0.068 | 0.053 | 0.059 |
| | (0.003) | (0.011) | (0.019) | (0.020) | (0.017) | (0.026) | (0.025) |
| pT231/T231 | 0.050 | 0.081 | 0.203 | 0.147 | 0.151 | 0.146 | 0.157 |
| | (0.013) | (0.046) | (0.069) | (0.061) | (0.051) | (0.065) | (0.055) |

AD, Alzheimer's disease; CN, cognitively normal; MMSE; Mini Mental State Exam; CDRSB; Clinical Dementia Rating Scale Sum of Boxes; ADAS13, Alzheimer's Disease Assessment Scale; RBANS; Repeatable Battery for the Assessment of Neuropsychological Status; PET, positron emission tomography; ROI, region of interest; SUVR, standardized uptake value ratio.

1.2. Cross-sectional Associations Between Tau Phosphosites and Amyloid and Tau PET Uptake: In the full-range cohort (cognitively normal through moderate AD), [¹⁸F]FBP uptake increased and plateaued in the AD patients, unlike [¹⁸F]GTP1, which increased in a 5 stepwise manner with disease severity (FIGS. 9A-B3). [¹⁸F]GTP1 offered greater differentiation between diagnostic groups than [¹⁸F]FBP, as evidenced by larger hedge effect sizes (G=0.38 and 0.02 between prodromal-moderate and mild-moderate for FBP versus G=1.17 and 0.56 between prodromal—moderate and mild—moderate for [¹⁸F]GTP1). P-tau181 monitored by immunoassay followed the same pattern as FBP (FIG. 9C-D). For the eleven phosphorylation sites monitored by MS (T111, T153, T175, T181, S199, S202, T205, S208, S214, T217, and T231), most of the sites displayed an increase in their phosphorylation rates (phosphorylated peptide/unphosphorylated peptide) (p-tau/u-tau) from amyloid-negative controls to prodromal stages (i.e., T181, T111, T153, S199, T205, S208, S214, T217 and T231) (FIGS. 9E-G, I, K-O). Sites T181, S214, and T231 (FIGS. 9E, M, and O) spiked in prodromal AD compared to control subjects, and then declined as disease progressed to mild AD and moderate AD. In other words, these sites show an increase in prodromal AD compared to control subjects, while the phosphosite level then declines as disease progresses from prodromal to mild and moderate AD. In the case of T217 (FIG. 9N), there was an increase in occupancy ratio of the phosphosites in prodromal AD subjects compared to control subjects, where the ratio then plateaus for mild AD and moderate AD in comparison to prodromal AD (i.e., it does not further increase in mild and moderate AD subjects compared to prodromal AD subjects). Sites T111, T153, S208, and T217 (FIGS. 9F, G, L, and N), like Elecsys® p-tau181, followed the amyloid PET pattern and plateaued with increasing AD severity, as did S199 (FIG. 9I), though the increase in phosphorylation was slight. Conversely, progressive decreases in phosphorylation rates were observed for T175 and S202 (FIGS. 9H and J) with increasing disease severity, analogous to the progressive increases in [¹⁸F]GTP1 PET uptake with increasing disease severity. T172 and S202 showed decreased levels in prodromal AD compared with control subjects and further decreased levels in mild and moderate AD subjects compared to prodromal AD subjects. The decrease observed for S202 is consistent with the finding that S202 is hypophosphorylated in disease rather than hyperphosphorylated.

None of the sites monitored resemble the step-wise increase observed for the [¹⁸F]GTP1 PET tracer with disease progression. While it can't be assessed in this cross-sectional cohort, this could indicate that the timing of these changes appears at different stages in CSF and in brain tissue. Cross-sectional patterns of phosphorylation rates at the other monitored phosphosites did not fit into the patterns seen with [¹⁸F]FBP or [¹⁸F]GTP1 PET. T181, S214, and T231 peaked in prodromal AD, and then decreased in mild and moderate AD. S199 and T205 showed a bell-shaped distribution that peaked in mild AD.

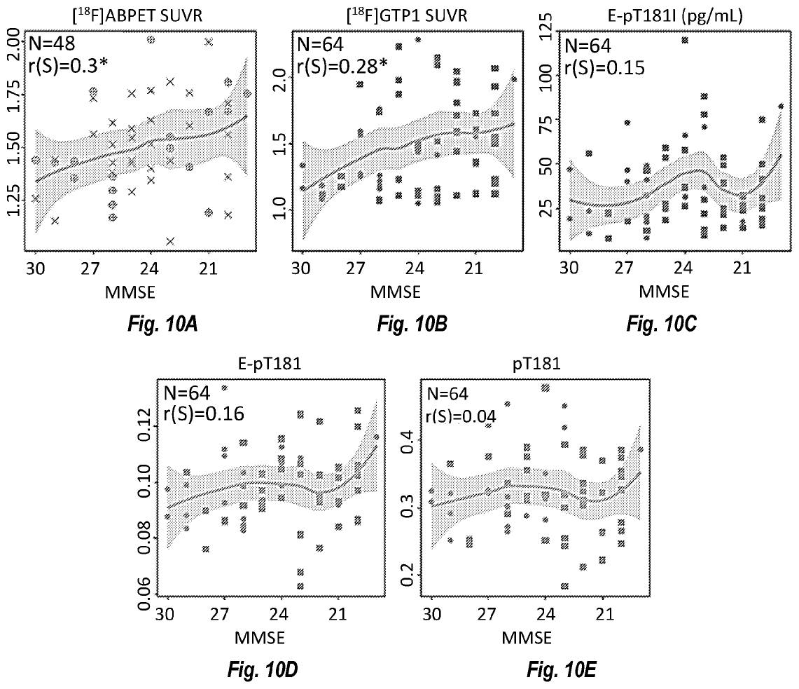

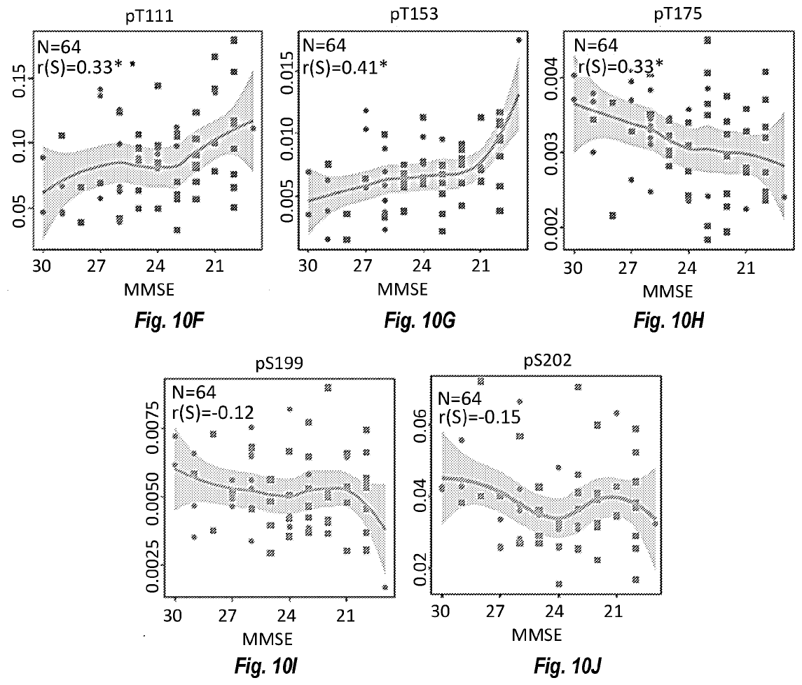

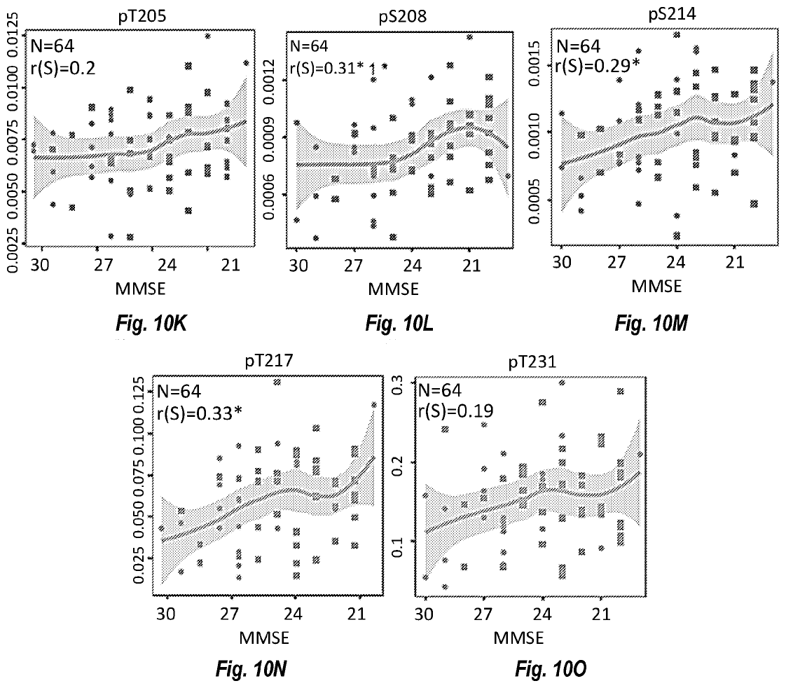

Diagnostic groups in the full range AD cohort were strongly separated by MMSE scores. To produce comparable figures for the early AD cohort, which only included two diagnostic groups, we show scatter plots with MMSE scores on the x-axis instead of boxplots with diagnostic groups. Similar trends to the full range AD cohort were observed in the early AD cohort with phosphorylation increasing with disease severity for the majority of sites, with the exception of T175, 5199 and S202 that were decreasing as cognitive performance declined (FIGS. 10A-O), however, no significant differences were observed between prodromal and mild patients.

1.3. CSF tau phosphosites cluster and patterns shift with disease severity: The Elecsys® immunoassay was used as a benchmark comparison for the MS assay. Because the MS assay uses ratios, we evaluated the performance of the Elecsys® p-tau181/t-tau ratio, which showed greater separation between CN and AD patients than Elecsys® p-tau181 levels (G=1.21; 1.12; 1.32 between CN Aβ+ and prod, mild, and mod respectively for p-tau/t-tau versus G=0.57; 0.68; 0.94 between CN Aβ+ and prod, mild, and mod respectively for p-tau). The Elecsys® p-tau181/t-tau and MS pT181/uT181 ratios correlated more strongly in the full range AD cohort (r(S)=0.73) than in the early AD cohort (r(S)=0.64). In FIGS. 11A-D, red color indicates phosphosites having levels that are positively correlated with Elecsys® pTau181/t-tau assay results (E_pTau/t Tau) and with results for other phosphosites; blue color indicates phosphosites whose levels are negatively correlated with Elecsys® pTau181/t-tau assay results (E_pTau/tTau) and with other phosphosite levels. Numbers shown are Spearman correlations. Levels for comparisons are calculated as occupancy ratios of phosphorylated levels to unphosphorylated peptide levels (e.g., pT217/T217 and so forth, as described above).

In both cohorts, T217 phosphorylation rates exhibited numerically higher correlations with Elecsys® p-tau181/t-tau ratios than MS phosphorylation rates (p-tau/u-tau) measured at other sites (FIGS. 11A-D). This association was particularly high at an earlier stage (r(S) 0.91 in full range AD CN-prod, FIG. 11B). The rise of T217 phosphorylation rates from cognitively normal to prodromal was highly associated with changes of phosphorylation observed on the other sites T153, T181, T111, S208, and T231. Together, these 6 phosphorylated tau sites could be grouped into a strong cluster (r(S) from 0.93 to 0.86, FIG. 11B). Correlations for this “pT217 cluster” were numerically weaker for participants at later symptomatic stages in both cohorts (FIGS. 11C-D), principally due to divergent trajectories for different sites from mild to moderate stages (plateau for 111, 153, 208, and 217, decrease for 181 and 231, FIGS. 9A-O).

T175 behaved independently from all other phosphorylation sites at all stages in both cohorts (−0.30<r(S)<0.36, FIG. 11A-D). Phosphorylation rates at S199 and S202 were moderately associated together across different disease stages in both cohorts (FIG. 11A-D). Phosphorylation at these two sites significantly decreased as phosphorylation at T217 increased in the prodromal-mild early AD cohort (FIG. 11D). Only S202 phosphorylation was inversely associated with T217 phosphorylation in the full range AD cohort. T205 phosphorylation associated with phosphorylation in the T217 cluster but also with S214 phosphorylation in cognitive normal-prodromal (FIG. 11B). This association with the pT217 cluster was significantly decreased in mild to moderate full range AD and prodromal to mild early populations (FIGS. 11C-D). For the prodromal to mild early AD population, we observed notable associations between pS208, pS214, and pT217 but not with other sites from the pT217 cluster. Association of pT205 with pS208 and pS214 remained observable in the mild to moderate group.

The pattern of phosphorylation observed for the multiple sites was more homogeneous in cognitive normal and prodromal AD subjects than for patients at more severe stages of the disease. While in the former subgroup, the first latent factor of the PCA captured 91% of the variance of the 11 phosphosites and Elecsys® p-tau181/t-tau phosphorylation ratio, the corresponding value was 66% in the mild to moderate subcohort.

1.4. Cross-sectional Association of CSF Tau Phosphosites [¹⁸F]GTP1 and [¹⁸F]FBP Uptake: FIGS. 12A-B show Spearman correlations between ¹⁸F GTP1 SUVR (standard uptake value ratio) in a few different ROIs (the temporal meta, whole cortical grey, and the Braak regions, grouped by 1-2, 3-4, and 5-6) and the levels of particular phosphosites (measured as ratios of phosphorylated tau peptide to total tau peptide) in full range AD (FIG. 12A) and early AD (FIG. 12B). Braak 1-2 SUVR is a location of tau particles often found in early stage AD, while Braak 5-6 is a location of tau particles often found in later stage AD. The figure also shows correlation with Elecsys® pTau181/t-tau (bottom line of tables). Across these different ROIs, there were consistent correlations for a handful of the phosphosites. Some phosphosites were positively correlated with ¹⁸F GTP1 (red), and a few phosphosites (pT175 and pS202) were negatively correlated (blue).

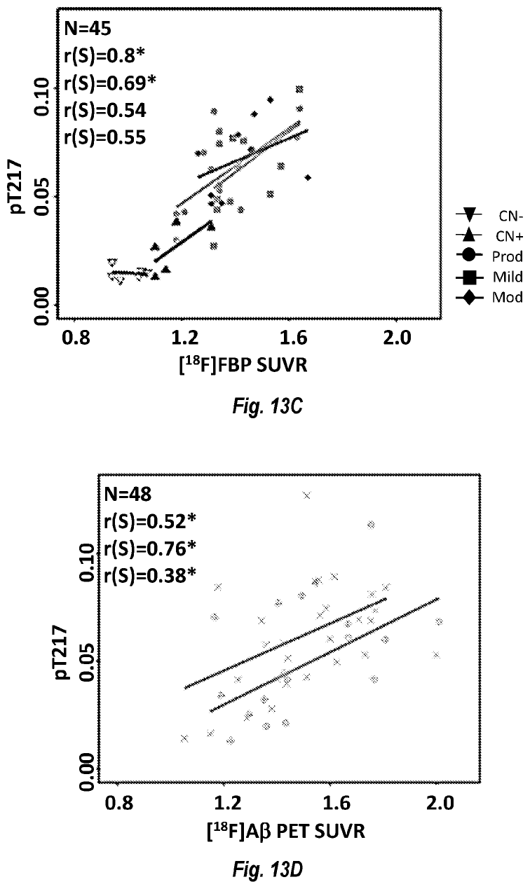

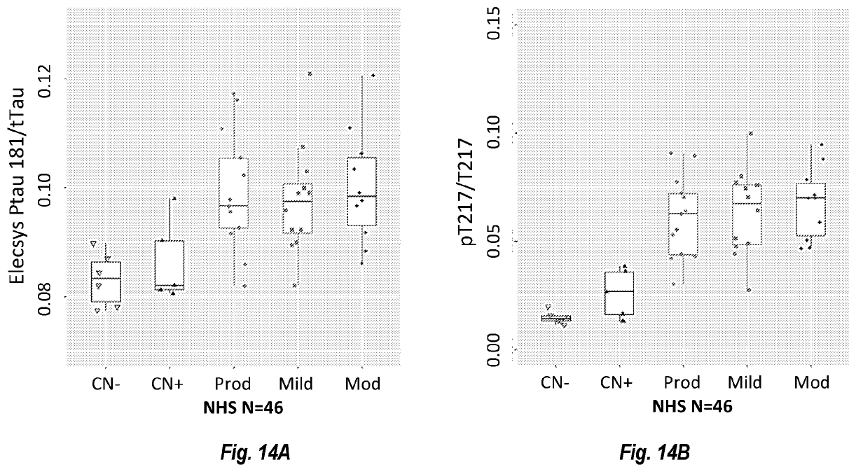

pT217 had the strongest association with [¹⁸F]GTP1 SUVR in both cohorts (FIGS. 12A-B). In prodromal AD (r(S)=0.83 [full-range AD cohort] and 0.87 [early AD cohort], FIGS. 13A-B), pT217 correlates well with [¹⁸F]FBP in Aβ+ subgroups of the full range AD cohort (FIG. 13C), but the association is weaker in the early AD cohort (FIG. 13D). [¹⁸F]GTP1 SUVR was elevated in moderate AD relative to the other cohorts whereas pT217 levels plateaued (FIGS. 9A-N). The association between [¹⁸F]GTP1 PET and [¹⁸F]Aβ PET SUVR is weaker (r(S)=0.69 [full range AD cohort] and 0.44 [early AD cohort], FIGS. 12A-B) than the association of either biomarker with pT217, implying that elevations in pT217 may become measurable in the temporal interval between the elevations observed with [¹⁸F]Aβ PET and [¹⁸F]GTP1 PET SUVRs. Other hyperphosphorylated sites were moderately associated with [¹⁸F]GTP1 SUVR in both cohorts. Conversely, phosphorylation rates at S202, S199, and T175 showed inverse correlations with [¹⁸F]GTP1 SUVR (FIGS. 12A-B and 13A-B). This trend was slightly stronger for pS202 and pT175 in the early AD cohort (FIGS. 12A-B and 13B). The multivariate linear model with LASSO identified three key sites (181, 205, and 217) as being the most associated with [¹⁸F]GTP1. Numerically, the model performance of this combination of sites was higher than 217 alone, with more marked differences seen in the full range AD cohort (R2=65% combined versus 55% 217 alone) than in the early AD cohort (R2=59% combined versus 57% 217 alone). Among the phosphorylated sites tested, pT217 was also most closely associated with [¹⁸F]Aβ PET SUVR (r(S)=0.80 [full range AD cohort]; r(S)=0.52 [early AD cohort], FIG. 13A-B).

Additional results are shown in FIGS. 13-16. FIGS. 13A-D show a closer look at the correlation plots for pT217 and [¹⁸F]GTP1 in the temporal meta ROI. Correlation of pT217 with [¹⁸F]GTP1 was replicated in prodromal to mild AD, and consistent correlations were identified across the board. (Correlations are Spearman correlations.)

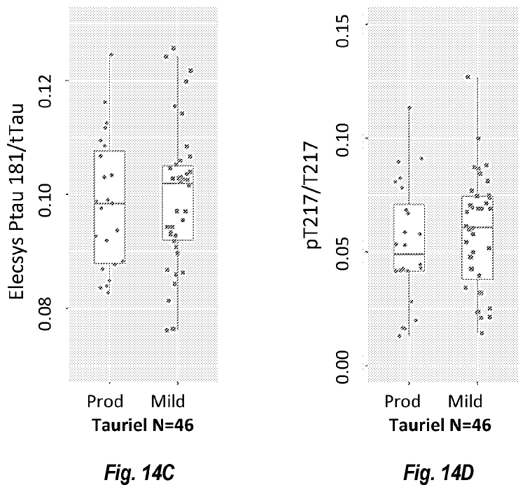

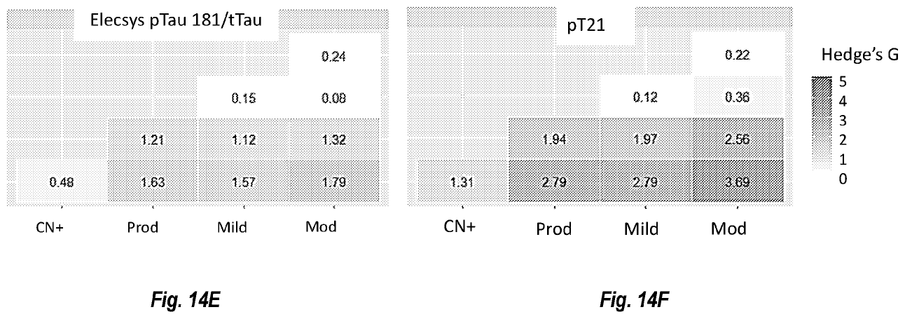

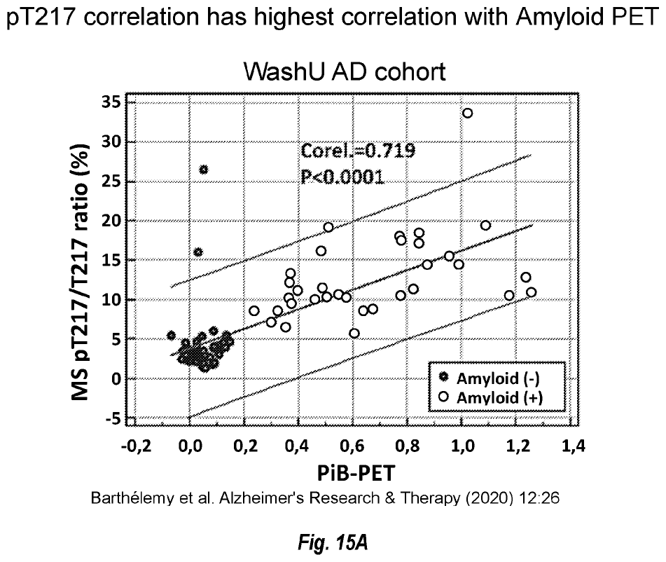

FIGS. 14A-F show that pT217/T217 offers a potentially greater differentiation between controls and AD, and that the differentiation may continue to improve through mild AD. Since pT217 outperformed the other sites with regards to correlation with ¹⁸F GTP1, we studied how its effect size compared with the classic measure of pT181. While the profiles are similar, the effect size analysis demonstrated that pT217 more clearly differentiates the diagnostic cohorts than pT181.

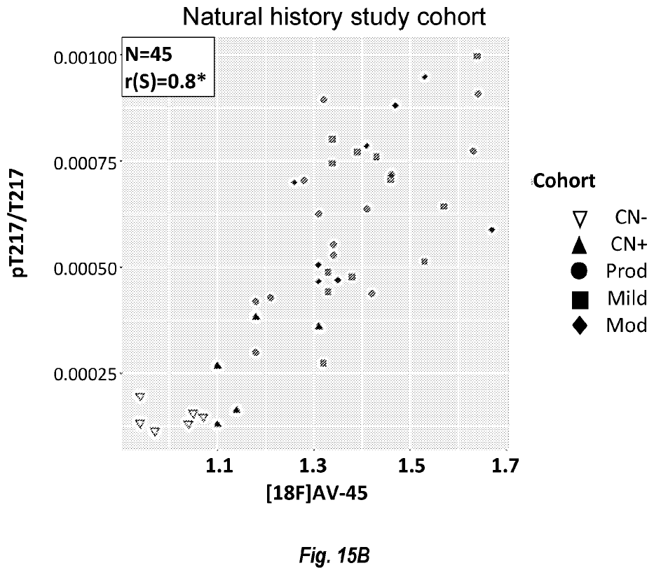

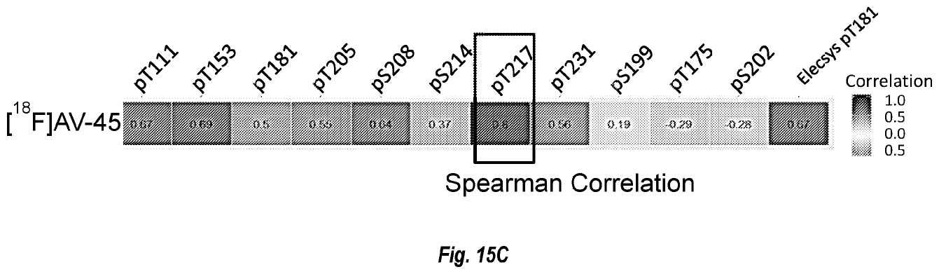

FIG. 15A-C show that the pT217/T217 correlation with amyloid beta PET is the highest of the various phosphosites (see the box in the table below the graphs). We followed up on a prior study that found that pT217 correlates with PiB-PET (Pittsburgh Compound B, an amyloid beta PET tracer). (In the upper left hand graph in FIG. 15, the Amyloid + circles are distributed to the right of 0.2 PiB-PET on the x-axis. The Amyloid − circles are distributed to the left of 0.2 PiB-PET on the x-axis.) Specifically, we repeated this analysis in our NHS/e0048 study cohorts, determining Spearman correlations between the phosphosite levels (measured as ratios of phosphorylated to total tau peptides) and values obtained from a different amyloid beta PET tracer, ¹⁸F AV-45 (also known as florbetapir). When we compared the performance of pT217 with that of the other phosphosites, we found that, while others also correlated with ¹⁸F AV-45, pT217 had the highest correlation. These data indicated that one or more tau phosphosites measured in CSF samples could be used as a close surrogate for amyloid beta PET imaging, such as ¹⁸F AV-45 PET imaging.

FIG. 16A-B show forest plots of Spearman correlations between phosphosite levels (measured as ratios of phosphorylated to total peptides) and each of 4 different cognitive assessments (MMSE, CDR-SB, ADAS13, and RBANS_TOTAL) performed on subjects in the NHS and Tauriel studies. (MMSE is described in Folstein, M F et al. “Mini-mental state”. A practical method for grading the cognitive state of patients for the clinician.” J Psychiatr Res. 1975 November; 12(3): 189-98. CDR-SB is described in Hughes, C P et al., “A new clinical scale for the staging of dementia.” Br J Psychiatry, 1982 June: 140: 566-72. ADAS13 is described in Skinner, J. et al., “The Alzheimer's Disease Assessment Scale-Cognitive-Plus (ADAS-Cog-Plus): an expansion of the ADAS-Cog to improve responsiveness in MCI.” Brain Imaging Behav. 6, 489-501(2012). RBANS_TOTAL is described in Randolph, C. et al., “The Repeatable Battery for the Assessment of Neuropsychological Status (RBANS): preliminary clinical validity.” J Clin Exp Neuropsychol, 1998 June; 20(3); 310-9. Each of these publications is incorporated by reference herein.)

Previously determined correlations between the cognitive assays and ¹⁸F GTP1 and Elecsys® pTau181 results are also presented, to serve as anchors. Solid shapes indicate statistical significance (P value of ≤0.05) while open (unfilled) shapes indicate lack of significance. Spearman correlations were used to assess relationships between the cognitive assessments and Tau either measured by GTP1 or CSF phosphorylation. Uncertainty in Spearman correlation estimates was characterized with 95% bootstrap confidence intervals. P-values and confidence intervals were not adjusted for multiple comparisons. ¹⁸F GTP1 values significantly correlated with all four assessments in the NHS/e0048 subjects. Similar to ¹⁸F GTP1, the pT111, pT205, and pT217 levels significantly correlated with the four cognitive assays in those subjects and to similar degrees. When we looked at the relationships in prodromal to mild AD, we found that ¹⁸F GTP1 results again significantly correlated with cognitive test results, in contrast to Elecsys® pTau181 results. In prodromal to mild AD subjects, pT217 level also significantly correlated to cognitive test results, and its correlation was numerically closest to that of ¹⁸F GTP1, as can be seen in analyzing the figure.

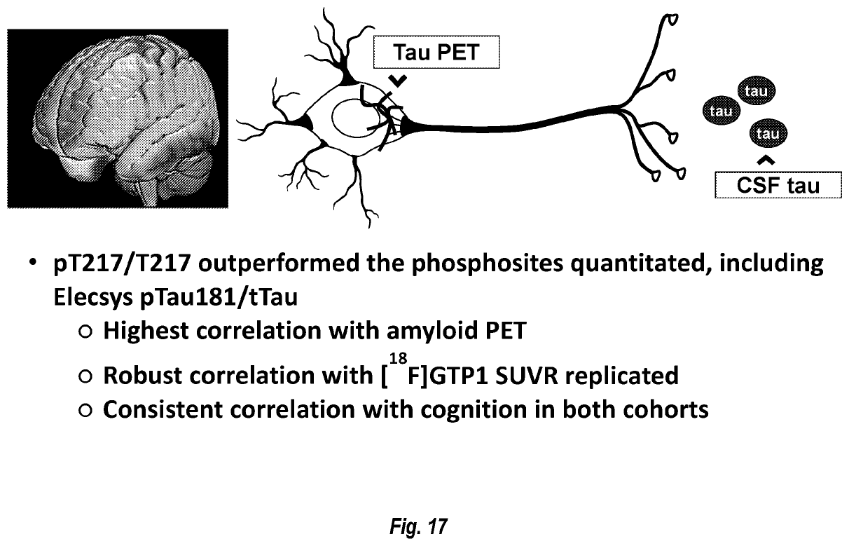

FIG. 17 summarizes some of the data presented herein, and describes that pT217/T217 correlated most strongly with ¹⁸F GTP1 in both the NHS/e0048 and Tauriel study cohorts, and more strongly than Elecsys® pTau181/tTau. In both study cohorts, pT217/T217 also had the strongest correlation with amyloid PET (¹⁸F AV-45), a robust correlation with [¹⁸F]GTP1 SUVR, and had a consistent correlation with cognitive assessments.

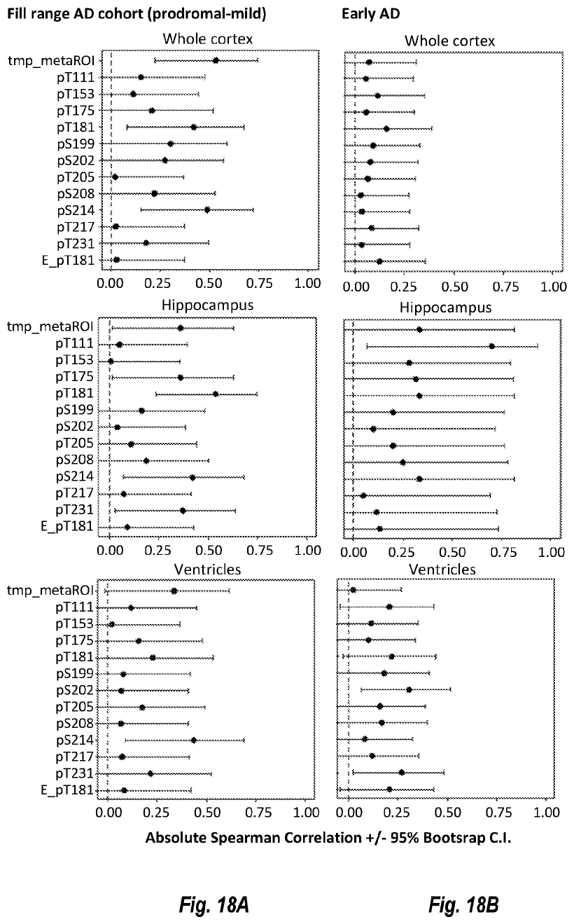

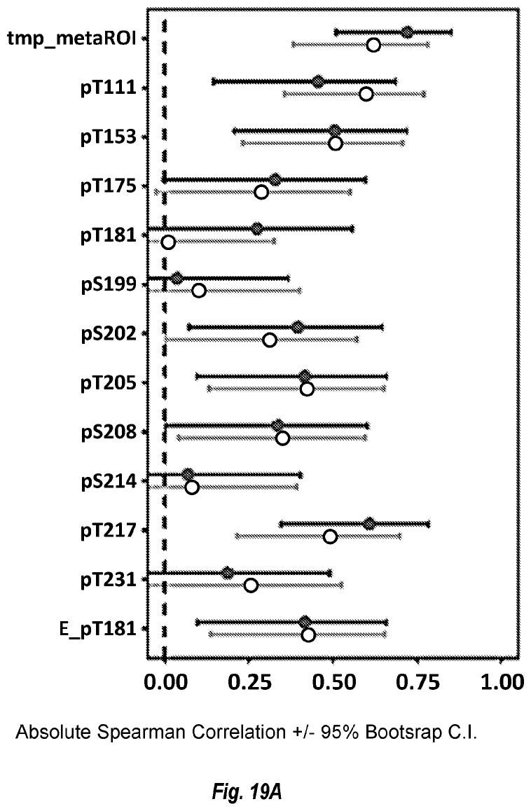

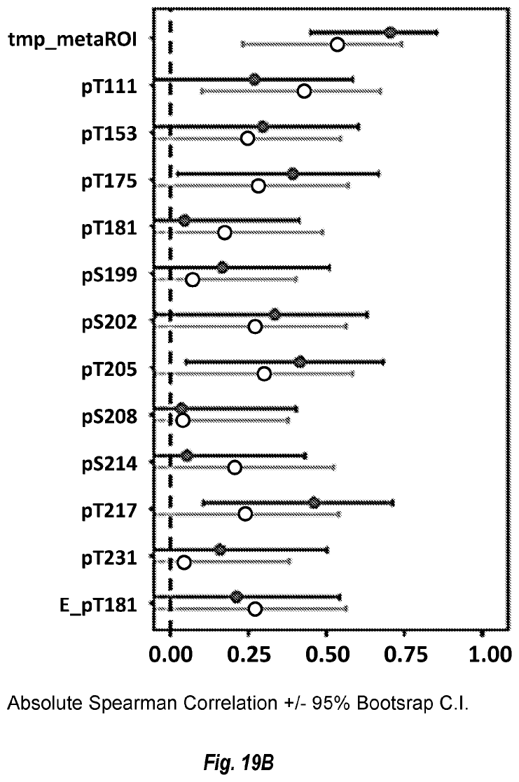

1.5. Association Between CSF Tau Phosphorylation, Tau PET and Brain Atrophy: We assessed the association between CSF tau phosphorylation, tau PET and brain atrophy measured by MRI in the two AD cohorts. The absolute Spearman correlations with confidence intervals (CI) for [¹⁸F]GTP1 SUVR in the temporal meta-ROI, the different phosphorylated species, whole cortical volume, hippocampal volume and ventricle volume is shown in FIG. 18A-B.

In the full range AD cohort (FIG. 18A), excluding cognitively normals, [¹⁸F]GTP1 SUVR had a significant correlation with whole cortical volume (r=0.53). pS214 was significantly correlated with whole cortex, hippocampal and ventricular volume. In the early AD cohort (FIG. 18B), pS202 and pT231 were weakly correlated with ventricular volume. pT111 had a significant correlation (r(S)=0.70 with hippocampal volume in the early AD cohort, but not the other measures tested in either cohort. None of the other sites nor [¹⁸F]GTP1 were significantly associated with brain volume in the early AD cohort FIG. 18B).

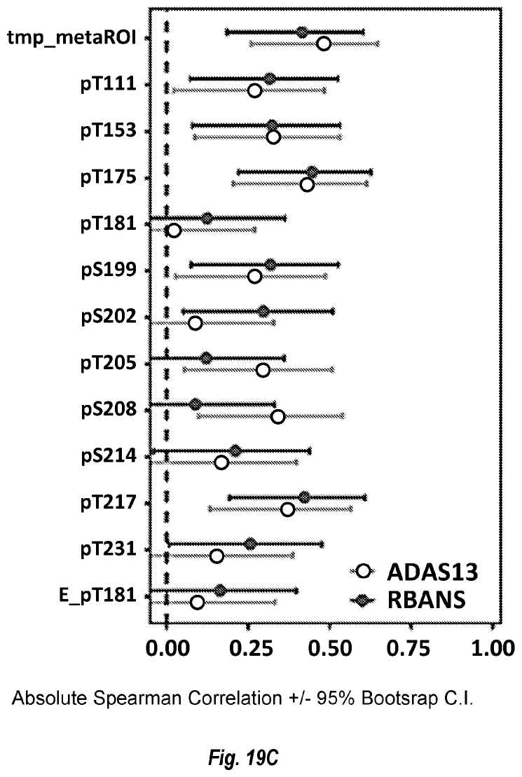

1.6. Association with Cognition: We compared the association of [¹⁸F]GTP1 SUVR and CSF p-tau with ADAS13, and RBANS cognitive measures (FIG. 19A-C). [¹⁸F]GTP1 SUVR was significantly and consistently associated with cognitive measures in the full range AD cohort (r(S)=0.62 with ADAS13 and r(S)=0.72 with RBANS FIG. 19A), consistent with previous reports (61), and this relationship replicated in the early AD cohort (r(S)=0.48 with ADAS13 and r(S)=0.42 with RBANS FIG. 19C). Of the 11 phosphosites monitored, the correlation of pT111, pT153 and pT217 with cognition closely resembled [¹⁸F]GTP1. The relationship weakened when cognitively-normal participants were removed (FIG. 13B), however, was significant again in the early AD cohort (FIG. 13C). pT175 correlated with cognition, similar to [¹⁸F]GTP1, on both tests in the early AD cohort (FIG. 13C). Other sites also had significant relationships, but the result was weaker and less consistent (pS202, pT205, pS208).

2. Discussion

CSF t-tau and p-tau181 levels have consistently demonstrated their utility for aiding in the diagnosis of Alzheimer's disease. However, because the levels of these analytes plateau at early stages of clinical disease, they have more limited utility as biomarkers of disease severity (72). Our study assessed tau phosphorylation across a wide range of phosphosites and stages of disease. We identified different patterns of associations between different CSF p-tau species and clinical disease severity, with some species plateauing or even decreasing in more advanced disease. These results suggest that ptau-181 may have better utility for monitoring asymptomatic AD and early AD, and other species, such as ptau-217 may be more useful for staging disease severity at symptomatic stages. Our results reveal different patterns of phosphorylation changes across the 11 investigated phosphosites. The comparison between MS results and Elecsys® p-tau181 and t-tau revealed slightly different profiles between the two techniques. While the overall correlation between these assays was robust, differences in the exact species that are captured by each assay [e.g., different antibodies used, presence of truncated species in CSF (29, 9)] can account for the observed differences in performance. We demonstrated phosphorylation rates at T217 have a higher association with both tau and Aβ PET. Our findings confirm previous reports suggesting stronger associations of phosphorylation at T217 than T181 with Aβ and tau PET measures (53, 54, 56). While phosphorylation at T181 was highly correlated with phosphorylation at other sites, such as T217, it was not as well correlated with disease pathology (AD and tau PET) or cognition. Our study reinforces the role of pT217 in more accurately reflecting the AD-related changes observed by brain imaging relative to the other CSF p-tau sites that have been examined. However, the diversity of phosphorylation trajectories associated with disease progression amongst these sites highlights the potential challenges associated with simply attributing increased CSF p-tau species in AD to increased aggregation and cerebral deposition of tau. [18F]GTP1 SUVR appears to gradually increase with disease severity and is more closely associated with brain atrophy and cognitive measures than CSF tau phosphorylation rates. Conversely, different patterns are observed for the majority of CSF p-tau sites with increasing clinical disease severity. Also, phosphorylation at some sites (T175, S202) decreased with disease severity, particularly amongst symptomatic participants. We hypothesize that the discrepancies between tau PET and CSF p-tau measures could result from two concomitant mechanisms affecting soluble tau phosphorylated sites in an opposite manner.

The first mechanism would imply that brain soluble tau becomes hyperphosphorylated in response to brain amyloid deposition and in absence of significant tau aggregation. In this scheme, tau isoforms that are released from neurons and detected in CSF are hyperphosphorylated in association to amyloid deposition. Such a relationship, which may be particularly sensitive for pT217, was previously reported in asymptomatic participants with positive Aβ PET scans in both late onset sporadic AD and early onset dominantly inherited AD (53, 56) and is supported by the results of our current analyses. The stronger association of pT217 compared to other investigated phosphorylated sites would support earlier or more sensitive change of pT217 in response to amyloidosis as previously suggested when compared to pT181, t-tau and pT205 (57). The relatively strong association with [18F]FBP of pT111, pT153 or pS208, as well as p-tau/t-tau ratio measured by Elecsys®, suggests that phosphorylation at these residues may also be detectable at preclinical stages of AD.

The similar patterns of signal saturation seen with [18F]FBP, pT217, pT111, pT153 and pS208 at later, symptomatic stages of disease severity (FIGS. 9A-O) also support the hypothesis that phosphorylation of certain CSF tau species may be more closely linked to cerebral Aβ deposition. Other phosphosites, such as T175 and S202, are not hyperphosphorylated in AD CSF and might be unaffected by pathological changes driven by amyloidosis. As such, the different trajectories on CSF tau phosphorylation across different phosphosites observed with increasing disease severity would support site-specific modification by different kinases and phosphatases (73). Thus, enzymatic pathways that affect only a subset of tau phosphosites might be dysregulated in conjunction with AD amyloidosis.

The second proposed mechanism involves a pool of hyperphosphorylated tau species becoming insoluble and accumulating into brain tau aggregates that are detectable by PET imaging. This process could in turn result in a reduction in the levels of corresponding phosphorylated isoforms in the soluble tau pool subsequently released into the CSF. In CSF samples from symptomatic DIAD patients, phosphorylation at T181 and T217 progressively decreases with increasing disease severity (57). Likewise, in our analyses of CSF from late-onset sporadic AD participants shows a similar pattern of plateauing and subsequently decreasing rates of phosphorylation at T181 after symptom onset. However, in our study cohort, unlike in DIAD, phosphorylation at T217 plateaued, but did not decrease after symptom onset. This difference may be related to the limited sensitivity of cross-sectional analyses (current cohort) relative to longitudinal analyses (prior DIAD studies).

Phosphosites, such as S199, T175, and S202, where progressive reductions in phosphorylation are seen with increasing [18F]GTP1 SUVR, we may hypothesize that changes in these species occur concomitantly to tau aggregation. Considering no further increases in phosphorylation rates for soluble CSF tau are observed after symptom onset, we would predict that phosphorylation at all 11 phosphosites analyzed here would be enriched in AD tau aggregates. Such a prediction is supported by differential phosphorylation analyses of brain soluble tau and AD tau aggregates (50, 73, 71).

Irrespective of mechanisms underlying these results, they suggest that the use and interpretation of CSF p-tau biomarkers for evaluating potential anti-tau therapeutics should be approached with caution. Indeed, longitudinal changes in tau pathology as measured by tau PET may be a straightforward and clinically applicable outcome measure compared to longitudinal changes in soluble p-tau species for evaluating impact of a potential tau-targeted treatment. However, phosphorylated CSF tau species may have greater utility for improving diagnostic accuracy, particularly at asymptomatic stages.

There are a number of factors that may limit the interpretation of our results, including the relatively modest sample size, the high proportion of participants who were amyloid-positive, and the cross-sectional nature of the study. Nevertheless, it is encouraging that observations in this study support prior observations from larger cohorts.

In summary, we evaluated tau phosphorylation rates across 11 different phosphosites previously associated with AD and identified different cross-sectional patterns that track with the clustering of these phosphosites with each other and disease pathology. Our results suggest that pT217 tracks with amyloid PET cross-sectionally, and interestingly also has the strongest correlation with [¹⁸F]GTP1 tau PET. There were no consistent relationships with brain volume, potentially because the majority of subjects were early AD. A few of the phosphosites, including pT217, consistently tracked with [¹⁸F]GTP1 demonstrating similar correlations with cognition. Future studies that include longitudinal observations, more AD participants at advanced stages of disease, and asymptomatic individuals at-risk for developing AD will increase our understanding of the performance of these fluid biomarkers of tau pathology and guide their use in clinical trials, potentially as alternatives to tau PET or as complementary prognostic markers of clinical progression.

3. Methods

3.1 Study Design: The full clinical range AD cohort drew from patients enrolled in two studies: (1) study e0048 (75) evaluated the basic performance and reproducibility characteristics of [¹⁸F]GTP1; and (2) study GN30009 (NCT02640092; 61) was an open-label observational study evaluating longitudinal change in [¹⁸F]GTP1 tau PET imaging in AD patients and cognitively normal (CN) controls. The early AD cohort included patients from a Phase II study that evaluated the efficacy and safety of semorinemab (GN39763; NCT03289143) in prodromal to mild Alzheimer's disease (61, 76-78). [¹⁸F]florbetapir (FBP) PET, MRI scans, CSF, and cognitive data were also acquired in these studies.

3.2 Participants: The full-range clinical cohort was composed of control, prodromal, mild, and moderate AD participants from GN30009 (NCT02640092) and e0048, as previously described (36, 61). AD patients were required to have a positive FBP Aβ PET scan by visual read. The early AD cohort from GN39763 (NCT03289143) included participants with early AD who were between 50-80 years old, had MMSE≥20, CDR=0.5 or 1, RBANS Delayed Memory Index≤85, and were Aβ positive by PET scan (FBP or florbetaben [FBP]; visual read) or CSF (Elecsys® Aβ42≤1000 μg/mL). Each study was approved by each site's institutional review board and was conducted in accordance with ICH E6 Guidelines. Written informed consent was obtained from all participants or their legal representatives.

3.3 CSF Collection and Analyses: CSF (up to 20 mL) was collected by lumbar puncture into polypropylene tubes, centrifuged at 2,000×g for 10 min at room temperature, and transferred into 0.5 mL tubes that were frozen and stored at −80° C. until analysis. CSF collection and [¹⁸F]GTP1 PET scans for the full AD cohort were separated by an average of 14.3±12.4 days (range: 0-64 days). CSF collection and [¹⁸F]GTP1 PET scans for the early AD cohort were separated by an average of 32.6±23.5 days (range: 0-111 days).

CSF tau species were captured by immunoprecipitation (IP) and analyzed using a high-resolution MS quantitating multiple tau phosphorylation sites and their corresponding unphosphorylated peptide (50). Tau from CSF (500 μl) was extracted and immunopurified with Tau1 and HJ8.5 antibodies as previously reported (9). Briefly, 15N tau labeled standard was added to CSF sample prior to immuno purification, and after overnight digestion with trypsin, AQUA peptides (Life Technologies, Carlsbad, Calif.) were spiked to obtain an amount of 5 fmol per labeled phosphorylated peptide and 50 fmol per labeled unmodified peptide in each sample. The peptide mixture was purified by solid phase extraction. Eluates were dried and resuspended in MS vials prior nanoLC-MS/HRMS analysis on nanoAcquity UPLC system (Waters, Mildford, Mass.) coupled to a Lumos Tribrid MS (Thermo Scientific, San Jose, Calif.) as reported (50).

MS/HRMS transitions were extracted using Skyline software (MacCoss lab, University of Washington). CSF tau phosphorylation levels were calculated using measured ratios between MS/HRMS transitions of endogenous unphosphorylated peptides and 15N labeled peptides from protein internal standard. Ratios of phosphorylation at the T181, S202, T205, and T217 sites were measured using the ratio of the MS/HRMS transitions from phosphorylated peptides (103-126 for pT111; 151-155 for pT153; 171-180 for pT175; 175-190 for pT181; 195-209 for pS202, pT205 and pS208; 212-221 for pS214 and pT217; 226-234 for pT231) and corresponding unphosphorylated peptides (103-126 for T111; 151-155 for T153; 181-190 for T181 and T175, 195-209 for S202, T205 and S208; 212-221 for S214 and T217; 226-230 for T231). Each phosphorylated/unphosphorylated peptide endogenous ratio was normalized using the ratio measured on the MS/HRMS transitions of the corresponding AQUA phosphorylated/unphosphorylated peptide internal standards. Ratios of phosphorylation at the T111, T153, 5208, and S214 sites were measured without internal standard using corresponding non-phosphorylated peptides signal as reference.

We also compared our quantitative MS results to p-tau181 levels and p-tau181/t-tau ratios using the Elecsys® in vitro diagnostics immunoassay (Roche Diagnostics, Penzberg, Germany), which captures mid-domain species containing residues 159-224 (t-tau) and 175-200 (p-tau181) (79).

3.4 Imaging: [¹⁸F]GTP1 synthesis and [¹⁸F]GTP1 and Aβ PET imaging was performed as previously described (37, 61). [¹⁸F]GTP1 SUVRs were calculated using the cerebellar gray as reference. ROIs included the whole cortical gray matter (WCG), a AD-specific temporal (TMP) meta-ROI (38) and hierarchical in vivo Braak tau PET stages (I-II, III-IV, V-VI; Schöll et al., 2016). Aβ PET was performed using [¹⁸F]FBP (GN30009, e0048 and GN39763) or [¹⁸F]FBB (GN39763) prepared at commercial facilities. Participants were defined as Aβ positive by visual read. For quantitative purposes, the cognitively normals (GN30009, e0048) were defined as Aβ positive if their [¹⁸F]FBP SUVR was above 1.10 in a composite cortical ROI, and Aβ negative if their FBP SUVR was below 1.10 (80). Aβ PET SUVRs were calculated using the whole cerebellum as reference. MRI was performed for participant eligibility (e.g., potential participants with evidence of other pathologies that might contribute to cognitive impairment were excluded), [¹⁸F]GTP1 and Aβ PET image processing, and volumetric analyses. Hippocampus, whole cortex and ventricle volume were normalized by the intracranial volume.

3.5 Statistical Analyses: Spearman correlations were used to assess relationships between different AD biomarkers and clinical assessments. Uncertainty in Spearman correlation estimates was characterized with 95% bootstrap confidence intervals. P-values and confidence intervals were not adjusted for multiple comparisons. Baseline means and standard deviations of demographic, cognitive, neuroimaging and CSF tau phosphorylation indices are reported. We calculated Hedges' g-s to compare the separation of clinical subgroups in the full range AD cohort by the measured biomarkers. CSF tau phosphorylation at various sites were clustered by correlation distance and represented by dendrograms. We assessed the similarity/dissimilarity of the phosphorylation ratios of subjects in subgroups of the full range AD cohort by principal component analysis (PCA), applying the singular value decomposition approach to the standardized features. Association between [¹⁸F]GTP1 SUVR and CSF phosphorylation ratios was also assessed in a multivariate linear regression model with LASSO penalty. The model performance was measured by adjusted R². R software version 3.5.2 (R Core Team, 2016) was used for all analyses.

### REFERENCES

1. Alzheimer's Disease Facts and Figures. https://www.alz.org/alzheimers-dementia/facts-figures. (2019).
2. H. Braak, E. Braak, Neuropathological stageing of Alzheimer-related changes. Acta Neuropathol 82, 239-259 (1991).
3. H. Braak, E. Braak, Staging of Alzheimer's disease-related neurofibrillary changes. Neurobiol Aging 16, 271-278; discussion 278-284 (1995).
4. M. J. Pontecorvo, M. D. Devous, Sr., M. Navitsky, M. Lu, S. Salloway, F. W. Schaerf, D. Jennings, A. K. Arora, A. McGeehan, N. C. Lim, H. Xiong, A. D. Joshi, A. Siderowf, M. A. Mintun, F. A.-A. investigators, Relationships between flortaucipir PET tau binding and amyloid burden, clinical diagnosis, age and cognition. Brain 140, 748-763 (2017).
5. C. R. Jack, Jr., H. J. Wiste, C. G. Schwarz, V. J. Lowe, M. L. Senjem, P. Vemuri, S. D. Weigand, T. M. Therneau, D. S. Knopman, J. L. Gunter, D. T. Jones, J. Graff-Radford, K. Kantarci, R. O. Roberts, M. M. Mielke, M. M. Machulda, R. C. Petersen, Longitudinal tau PET in ageing and Alzheimer's disease. Brain 141, 1517-1528 (2018).
6. A. Maass, S. Landau, S. L. Baker, A. Horng, S. N. Lockhart, R. La Joie, G. D. Rabinovici, W. J. Jagust, I. Alzheimer's Disease Neuroimaging, Comparison of multiple tau-PET measures as biomarkers in aging and Alzheimer's disease. Neuroimage 157, 448-463 (2017).
7. E. D. Roberson, K. Scearce-Levie, J. J. Palop, F. Yan, I. H. Cheng, T. Wu, H. Gerstein, G. Q. Yu, L. Mucke, Reducing endogenous tau ameliorates amyloid beta-induced deficits in an Alzheimer's disease mouse model. Science 316, 750-754 (2007).
8. H. Braak, K. Del Tredici, The preclinical phase of the pathological process underlying sporadic Alzheimer's disease. Brain 138, 2814-2833 (2015).
9. C. Sato, N. R. Barthelemy, K. G. Mawuenyega, B. W. Patterson, B. A. Gordon, J. Jockel-Balsarotti, M. Sullivan, M. J. Crisp, T. Kasten, K. M. Kirmess, N. M. Kanaan, K. E. Yarasheski, A. Baker-Nigh, T. L. S. Benzinger, T. M. Miller, C. M. Karch, R. J. Bateman, Tau Kinetics in Neurons and the Human Central Nervous System. Neuron 97, 1284-1298 e1287 (2018).
10. S. Takeda, S. Wegmann, H. Cho, S. L. DeVos, C. Commins, A. D. Roe, S. B. Nicholls, G. A. Carlson, R. Pitstick, C. K. Nobuhara, I. Costantino, M. P. Frosch, D. J. Muller, D. Irimia, B. T. Hyman, Neuronal uptake and propagation of a rare phosphorylated high-molecular-weight tau derived from Alzheimer's disease brain. Nat Commun 6, 8490 (2015).
11. Y. Wang, E. Mandelkow, Tau in physiology and pathology. Nat Rev Neurosci 17, 5-21 (2016).
12. J. W. Wu, S. A. Hussaini, I. M. Bastille, G. A. Rodriguez, A. Mrejeru, K. Rilett, D. W. Sanders, C. Cook, H. Fu, R. A. Boonen, M. Herman, E. Nahmani, S. Emrani, Y. H. Figueroa, M. I. Diamond, C. L. Clelland, S. Wray, K. E. Duff, Neuronal activity enhances tau propagation and tau pathology in vivo. Nat Neurosci 19, 1085-1092 (2016).
13. S. K. Herukka, J. Rummukainen, J. Ihalainen, M. von Und Zu Fraunberg, A. M. Koivisto, O. Nerg, L. K. Puli, T. T. Seppala, H. Zetterberg, O. T. Pyykko, S. Helisalmi, H. Tanila, I. Alafuzoff, M. Hiltunen, J. Rinne, H. Soininen, J. E. Jaaskelainen, V. Leinonen, Amyloid-beta and Tau Dynamics in Human Brain Interstitial Fluid in Patients with Suspected Normal Pressure Hydrocephalus. J Alzheimers Dis 46, 261-269 (2015).
14. S. Magnoni, T. J. Esparza, V. Conte, M. Carbonara, G. Carrabba, D. M. Holtzman, G. J. Zipfel, N. Stocchetti, D. L. Brody, Tau elevations in the brain extracellular space correlate with reduced amyloid-beta levels and predict adverse clinical outcomes after severe traumatic brain injury. Brain 135, 1268-1280 (2012).
15. T. Sunderland, G. Linker, N. Mirza, K. T. Putnam, D. L. Friedman, L. H. Kimmel, J. Bergeson, G. J. Manetti, M. Zimmermann, B. Tang, J. J. Bartko, R. M. Cohen, Decreased beta-amyloid1-42 and increased tau levels in cerebrospinal fluid of patients with Alzheimer disease. JAMA 289, 2094-2103 (2003).
16. K. Yamada, J. R. Cirrito, F. R. Stewart, H. Jiang, M. B. Finn, B. B. Holmes, L. I. Binder, E. M. Mandelkow, M. I. Diamond, V. M. Lee, D. M. Holtzman, In vivo microdialysis reveals age-dependent decrease of brain interstitial fluid tau levels in P301S human tau transgenic mice. J Neurosci 31, 13110-13117 (2011).
17. M. Goedert, D. S. Eisenberg, R. A. Crowther, Propagation of Tau Aggregates and Neurodegeneration. Annu Rev Neurosci 40, 189-210 (2017).
18. A. Mudher, M. Colin, S. Dujardin, M. Medina, I. Dewachter, S. M. Alavi Naini, E. M. Mandelkow, E. Mandelkow, L. Buee, M. Goedert, J. P. Brion, What is the evidence that tau pathology spreads through prion-like propagation? Acta Neuropathol Commun 5, 99 (2017).
19. G. S. Gibbons, V. M. Y. Lee, J. Q. Trojanowski, Mechanisms of Cell-to-Cell Transmission of Pathological Tau: A Review. JAMA Neurol 76, 101-108 (2019).
20. A. de Calignon, M. Polydoro, M. Suarez-Calvet, C. William, D. H. Adamowicz, K. J. Kopeikina, R. Pitstick, N. Sahara, K. H. Ashe, G. A. Carlson, T. L. Spires-Jones, B. T. Hyman, Propagation of tau pathology in a model of early Alzheimer's disease. Neuron 73, 685-697 (2012).
21. M. Iba, J. L. Guo, J. D. McBride, B. Zhang, J. Q. Trojanowski, V. M. Lee, Synthetic tau fibrils mediate transmission of neurofibrillary tangles in a transgenic mouse model of Alzheimer's-like tauopathy. J Neurosci 33, 1024-1037 (2013).
22. L. Liu, V. Drouet, J. W. Wu, M. P. Witter, S. A. Small, C. Clelland, K. Duff, Trans-synaptic spread of tau pathology in vivo. PLoS One 7, e31302 (2012).
23. D. W. Sanders, S. K. Kaufman, S. L. DeVos, A. M. Sharma, H. Mirbaha, A. Li, S. J. Barker, A. C. Foley, J. R. Thorpe, L. C. Serpell, T. M. Miller, L. T. Grinberg, W. W. Seeley, M. I. Diamond, Distinct tau prion strains propagate in cells and mice and define different tauopathies. Neuron 82, 1271-1288 (2014).
24. H. Braak, I. Alafuzoff, T. Arzberger, H. Kretzschmar, K. Del Tredici, Staging of Alzheimer disease-associated neurofibrillary pathology using paraffin sections and immunocytochemistry. Acta Neuropathol 112, 389-404 (2006).
25. Kim W, Lee S, Hall G F. Secretion of human tau fragments resembling CSF-tau in Alzheimer's disease is modulated by the presence of the exon 2 insert. FEBS Lett 50 2010; 584:3085-8.
26. Taniguchi-Watanabe S, Arai T, Kametani F, Nonaka T, Masuda-Suzukake M, Tarutani A, et al. Biochemical classification of tauopathies by immunoblot, protein sequence and mass spectrometric analyses of sarkosyl-insoluble and trypsin-resistant tau. Acta Neuropathol 2016; 131: 267-80.
27. Ishiguro K, Ohno H, Arai H, Yamaguchi H, Urakami K, Park J M, et al. Phosphorylated tau in human cerebrospinal fluid is a diagnostic marker for Alzheimer's disease. Neurosci Lett 1999; 270: 91-4.
28. Borroni B, Gardoni F, Pametti L, Magno L, Malinverno M, Saggese E, et al. Pattern of Tau forms in CSF is altered in progressive supranuclear palsy. Neurobiol Aging 2009; 30:34-40.
29. Meredith J E Jr, Sankaranarayanan S, Guss V, Lanzetti A J, Berisha F, Neely R J, et al. Characterization of novel CSF Tau and ptau biomarkers for Alzheimer's disease. PLoS ONE 2013; 8: e76523.
30. Amadoro G, Corsetti V, Sancesario G M, Lubrano A, Melchiorri G, Bernardini S, et al. Cerebrospinal fluid levels of a 20-22 kDa NH2 fragment of human tau provide a novel neuronal injury biomarker in Alzheimer's disease and other dementias. J Alzheimer's Dis 2014; 42: 211-26.
31. Cicognola C, Brinkmalm G, Wahlgren J, Portelius E, Gobom J, Cullen N C, et al. Novel tau fragments in cerebrospinal fluid: relation to tangle pathology and cognitive decline in Alzheimer's disease. Acta Neuropathol 2019; 137: 279-96.
32. Arai T, Ikeda K, Akiyama H, Nonaka T, Hasegawa M, Ishiguro K, et al. Identification of amino-terminally cleaved tau fragments that distinguish progressive supranuclear palsy from corticobasal degeneration. Ann Neurol 2004; 55: 72-9.
33. Zhao X, Kotilinek L A, Smith B, Hlynialuk C, Zahs K, Ramsden M, et al. Caspase-2 cleavage of tau reversibly impairs memory. Nat Med 2016; 22: 1268-76.
34. Chen H H, Liu P, Auger P, Lee S H, Adolfsson O, Rey-Bellet L, et al. Calpain-mediated tau fragmentation is altered in Alzheimer's disease progression. Sci Rep 2018; 8:16725.
35. Sokolow S, Henkins K M, Bilousova T, Gonzalez B, Vinters H V, Miller C A, et al. Pre-synaptic C-terminal truncated tau is released from cortical synapses in Alzheimer's disease. J Neurochem 2015; 133: 368-79.
36. Sanabria Bohorquez S, Marik J, Ogasawara A, Tinianow J N, Gill H S, Barret O, et al. [18F]GTP1 (Genentech Tau Probe 1), a radioligand for detecting neurofibrillary tangle tau pathology in Alzheimer's disease. Eur J Nucl Med Mol Imaging 2019; 46: 2077-2089.
37. Sanabria-Bohorquez S, Marik J, Ogasawara A, Tinianow J N, Gill H S, Barret O. First-in-human evaluation of [18F]GTP1 (Genentech Tau Probe 1), a radioligand for detecting neurofibrillary tangle tau pathology in Alzheimer's disease. Eur J Nucl Med Mol Imaging 2019; 46: 2077-89.
38. Jack C R Jr, Wiste H J, Weigand S D, Themeau T M, Lowe V J, Knopman D S, et al. Defining imaging biomarker cut points for brain aging and Alzheimer's disease. Alzheimer's Dement 2017; 13: 205-16.
39. Blennow K, Wallin A, Agren H, Spenger C, Siegfried J, Vanmechelen E. Tau protein in cerebrospinal fluid: a biochemical marker for axonal degeneration in Alzheimer disease? Mol Chem Neuropathol 1995; 26: 231-45.
40. Barthélemy NR1, Mallipeddi N1, Moiseyev P1, Sato C1, Bateman R J, Tau Phosphorylation Rates Measured by Mass Spectrometry Differ in the Intracellular Brain vs. Extracellular Cerebrospinal Fluid Compartments and Are Differentially Affected by Alzheimer's Disease. Front Aging Neurosci. 2019 May 21; 11:121. doi: 10.3389/fnagi.2019.00121. eCollection 2019
41. Nelson P T, Alafuzoff I, Bigio E H, et al. Correlation of Alzheimer disease neuropathologic changes with cognitive status: a review of the literature. J Neuropath Exp Neurol 2012; 71:362-81.
42. Querfurth H W, LaFerla F M. Alzheimer's Disease. N Engl J Med 2010; 362:329-344.
43. Hanger D P, Anderton B H, Noble W. Tau phosphorylation: the therapeutic challenge for neurodegenerative disease. Trends Mol Med 2009; 15:112-9.
44. Despres C, Byrne C, Qi H, et al. Identification of the Tau phosphorylation pattern that drives its aggregation. Proc Natl Acad Sci 2017; 114(34):9080-9085.
45. Augustinack J C, Schneider A, Mandelkow E M, et al. Specific tau phosphorylation sites correlate with severity of neuronal cytopathology in Alzheimer's disease. Acta Neuropathol 2002; 103(1):26-3.
46. Blennow K, Hampel H. Cerebrospinal fluid markers for incipient Alzheimer's disease. Lancet Neurol 2003; 2:605-613.
47. Blennow K, Zetterberg H. The past and the future of Alzheimer's disease fluid biomarkers. J Alzheimers Disease 2018; 62:1125-1140.
48. Benzinger T L, Gordon B A, Morris J C, et al. Time course of amyloid and tau deposition in autosomal dominant Alzheimer's disease: update from the Dominantly Inherited Alzheimer Network (DIAN) and DIAN-T U. Alzheimer Association International Conference; 2017.
49. Quiroz Y T, Sperling R A, Norton D J, et al. Association between amyloid and tau accumulation in young adults with autosomal dominant Alzheimer disease. JAMA Neurol 2018; 75(5):548-556.
50. Barthélemy N R, Mallipeddi N, Moiseyev P, et al. Tau Phosphorylation Rates Measured by Mass Spectrometry Differ in the Intracellular Brain vs. Extracellular Cerebrospinal Fluid Compartments and Are Differentially Affected by Alzheimer's Disease. Front Aging Neurosci 2019; 11:121.
51. Barthelemy N R, Gabelle A, Sato C, et al. Highly specific modification of tau phosphorylation stoichiometry in A D CSF impacts T217, S199, S202 and T205 sites but not T181. J Prev Alz Dis 2016; 4:295-296.
52. Barthélemy N R, Bateman R J, et al. Tau hyperphosphorylation on T217 in cerebrospinal fluid is specifically associated to amyloid-β pathology. bioRxiv 226977; doi: https://doi.org/10.1101/226977
53. Barthélemy N R, Bateman R J, Hirtz C, et al. Cerebrospinal fluid phospho-tau T217 outperforms T181 as a biomarker for the differential diagnosis of Alzheimer's disease and PET amyloid-positive patient identification. Alzheimers Res Ther 2020; 12(1):26.
54. Janelidze S, Stomrud E, Smith R, et al. Cerebrospinal fluid p-tau217 performs better than p-tau181 as a biomarker of Alzheimer's disease. Nat Commun 2020; 11(1):1683.
55. Palmqvist S, Janelidze S, Quiroz Y T, et al. Discriminative Accuracy of Plasma Phospho-tau217 for Alzheimer Disease vs Other Neurodegenerative Disorders.

JAMA 2020; 324(8):772-781.
56. Barthélemy N R, Horie K, Sato C, et al. Blood plasma phosphorylated-tau isoforms track CNS change in Alzheimer's disease. J Exp Med 2020; 217(11):e20200861.
57. Barthélemy N R, Li Y, Joseph-Mathurin N, et al. Dominantly Inherited Alzheimer Network. A soluble phosphorylated tau signature links tau, amyloid and the evolution of stages of dominantly inherited Alzheimer's disease. Nat Med 2020; 26(3):398-407.
58. Aschenbrenner A J, Gordon B A, Benzinger TLS, et al. Influence of tau PET, amyloid PET, and hippocampal volume on cognition in Alzheimer disease. Neurology 2018; 91(9):e859-e866.
59. Pontecorvo M J, Devous M D, Kennedy I, et al. A multicentre longitudinal study of flortaucipir (18F) in normal ageing, mild cognitive impairment and Alzheimer's disease dementia. Brain 2019; 142(6):1723-1735.
60. Sperling R A, Mormino E C, Schultz A P, et al. The impact of amyloid-beta and tau on prospective cognitive decline in older individuals. Ann Neurol 2019; 85(2):181-193.
61. Teng E, Ward M, Manser P T, et al. Cross-sectional associations between [¹⁸F]GTP1 tau PET and cognition in Alzheimer's disease. Neurobiology of Aging 2019; 81:138-145.
62. Betthauser T J, Koscik R L, Jonaitis E M, et al. Amyloid and tau imaging biomarkers explain cognitive decline from late middle-age. Brain 2020; 143(1):320-335.
63. Malpetti M, Kievit R A, Passamonti L, et al. Microglial activation and tau burden predict cognitive decline in Alzheimer's disease. Brain 2020; 143(5):1588-1602.
64. Gordon B A, Blazey T M, Su Y, et al. Spatial patterns of neuroimaging biomarker change in individuals from families with autosomal dominant Alzheimer's disease: a longitudinal study. Lancet Neurol 2018; 17(3):241-250.
65. Wang G, Xiong C, McDade E M, et al. Dominantly Inherited Alzheimer Network (DIAN). Simultaneously evaluating the effect of baseline levels and longitudinal changes in disease biomarkers on cognition in dominantly inherited Alzheimer's disease. Alzheimers Dement 2018; 4:669-676.
66. Binette A, Vachon-Presseau É, Morris J, et al. Amyloid and Tau Pathology Associations With Personality Traits, Neuropsychiatric Symptoms, and Cognitive Lifestyle in the Preclinical Phases of Sporadic and Autosomal Dominant Alzheimer's Disease. Biol Psychiatry 2020:S0006-3223(20) 30058-5.
67. Brier M R, Gordon B, Friedrichsen K, et al. Tau and Aβ imaging, CSF measures, and cognition in Alzheimer's disease. Sci Transl Med 2016; 8(338):338ra66.
68. Chhatwal J P, Schultz A P, Marshall G A, et al. Temporal T807 binding correlates with CSF tau and phospho-tau in normal elderly. Neurology 2016; 87(9):920-6.
69. Gordon B A, Blazey T, Su Y, et al. Longitudinal beta-amyloid deposition and hippocampal volume in preclinical Alzheimer disease and suspected non-Alzheimer disease pathophysiology. JAMA Neurol 2016; 73:1192-200.
70. Mattsson N, Insel P S, Palmqvist S, et al. Cerebrospinal fluid tau, neurogranin, and neurofilament light in Alzheimer's disease. EMBO Mol Med 2016; 8(10):1184-1196.
71. Lim Y, Maruff P, Johnson K, et al. Distinct information from CSF tau and AV1451 PET measures in nondemented individuals. Human Amyloid Imaging Conference, 2018.
72. McDade E, Wang G, Gordon B A, et al. Dominantly Inherited Alzheimer Network. Longitudinal cognitive and biomarker changes in dominantly inherited Alzheimer disease. Neurology 2018; 91(14):e1295-e1306.
73. Hanger D P, Byers H L, Wray S, et al. Novel phosphorylation sites in tau from Alzheimer brain support a role for casein kinase 1 in disease pathogenesis. J Biol Chem 2007; 282(32):23645-54.
74. Hone K, Barthelemy N R, Mallipeddi N et al. Regional correlation of biochemical measures of amyloid and tau phosphorylation in the brain. Acta Neuropathol. Commun. 2020; 8(1): 1-14
75. Ward M, Sanabria-Bohorquez S, Manser P T, et al. Cross-sectional associations between tau pathology burden measured by [18F]GTP1 PET imaging and cognition in Alzheimer's disease. J Prev Alz Dis 2017; 7(4).
76. Weimer R, Sanabria-Bohorquez S, Baker S L, et al. Longitudinal changes in [18F]GTP1 SUVR and cognition correlate with baseline tau burden and spatial distribution. Clinical Trials on Alzheimer's Disease, 2019.
77. Manser P T, Teng E, Pickthorn K, et al. Baseline correlations between [18F]GTP1 PET SUVR and MRI white matter hyperintensities in prodromal-to-mild Alzheimer's disease suggest independent contributions to cognitive impairment. Alzheimer's Association International Conference, 2020.
78. Teng E, Manser P T, Pickthorn K, et al. Cognitive indices correlate with [18F]GTP1 tau PET signal and white matter hyperintensities in prodromal to mild Alzheimer's disease: Baseline data from the Tauriel study. Alzheimer's Association International Conference, 2020.
79. Schindler S E, Gray J D, Gordon B A, et al. Cerebrospinal fluid biomarkers measured by Elecsys assays compared to amyloid imaging. Alzheimers Dement 2018; 14(11):1460-1469.
80. Landau S M, Breault C, Joshi A D, et al. Amyloid-beta imaging with Pittsburgh compound B and florbetapir: comparing radiotracers and quantification methods. J Nucl Med 2013; 54(1):70-7.

What is claimed is:

1. A method for determining whether a human subject is likely to have Alzheimer's disease (AD), comprising:
a. detecting the level of pT217 in a cerebrospinal fluid (CSF) sample from the subject and correlating the level of pT217 in the sample with a tau PET SUVR (standard uptake value rate) for the subject, such as a tau ([¹⁸F]GTP1) PET SUVR; and
b. determining whether the subject is likely to have AD based on the level of the pT217 compared to the tau PET SUVR, wherein the level of pT217 positively correlates with tau PET SUVR.

2. The method of claim 1, further comprising detecting the level of at least one further tau phosphosite in a cerebrospinal fluid (CSF) sample from the subject comprising (a) one or more of pT111, pT153, and pT175; (b) one or both of pS214 and pT231; (c) one or more of pT111, pT153, pT175, pS199, pS208, pS214, and pT231; (d) four or more of pT111, pT153, pT181, pS208, and pT231; or (e) one or more of pS199, pT175, and pS202.

3. The method of claim 1 or 2, further comprising determining whether the subject is likely to have AD based on the level of the pT217 compared to the tau PET SUVR, wherein the level of pT217 positively correlates with tau PET SUVR, and further based on the levels of (a) one or more of pT111, pT153, and pT175; (b) one or both of pS214 and pT231; (c) one or more of pT111, pT153, pT175, pS199, pS208, pS214, and pT231; (d) four or more of pT111, pT153, pT181, pS208, and pT231; or (e) one or more of pS199, pT175, and pS202, wherein levels of pT175 and pS202 decrease with increasing AD stage, levels of pT111, pT153, pT181, pS199, pS208, pS214, pT217 and pT231 are higher in AD subjects than in CN subjects, and levels of pS214 and pT231 are higher in prodromal AD subjects than in CN subjects but lower in mild and moderate AD subjects than in prodromal AD subjects.

4. The method of claim 1, 2, or 3, wherein the method is for determining whether the human subject is likely to have prodromal AD.

5. The method of claim 1, 2, or 3, wherein the method is for determining whether the human subject is likely to have mild AD.

6. The method of claim 1, 2, or 3, wherein the method is for determining whether the human subject is likely to have moderate AD.

7. A method of staging a human subject as likely to be cognitively normal (CN) or to have prodromal, mild, or moderate Alzheimer's disease (AD), comprising:
a. detecting a level of pT217 in a cerebrospinal fluid (CSF) sample from the subject and correlating the level of pT217 in the sample with a tau PET SUVR (standard uptake value rate) for the subject, such as a tau ([¹⁸F]GTP1) PET SUVR; and
b. determining whether the subject is likely to have AD based on the level of the pT217 compared to the tau PET SUVR, wherein the level of pT217 positively correlates with tau PET SUVR.

8. The method of claim 7, further comprising detecting the level of at least one further tau phosphosite in a cerebrospinal fluid (CSF) sample from the subject comprising (a) one or more of pT111, pT153, and pT175; (b) one or both of pS214 and pT231; (c) one or more of pT111, pT153, pT175, pS199, pS208, pS214, and pT231; (d) four or more of pT111, pT153, pT181, pS208, and pT231; or (e) one or more of pS199, pT175, and pS202.

9. The method of claim 7 or 8, further comprising determining whether the subject is likely to have AD based on the level of the pT217 compared to the tau PET SUVR, wherein the level of pT217 positively correlates with tau PET SUVR, and further based on the levels of (a) one or more of pT111, pT153, and pT175; (b) one or both of pS214 and pT231; (c) one or more of pT111, pT153, pT175, pS199, pS208, pS214, and pT231; (d) four or more of pT111, pT153, pT181, pS208, and pT231; or (e) one or more of pS199, pT175, and pS202, wherein levels of pT175 and pS202 decrease with increasing AD stage, levels of pT111, pT153, pT181, pS199, pS208, pS214, pT217 and pT231 are higher in AD subjects than in CN subjects, and levels of pS214 and pT231 are higher in prodromal AD subjects than in CN subjects but lower in mild and moderate AD subjects than in prodromal AD subjects.

10. The method of any one of claims 1-9, wherein the tau PET SUVR is tau ([¹⁸F]GTP1) PET SUVR.

11. The method of any one of claims 1-10, wherein the level of at least one of pS199, pT175, and pS202 is correlated with the tau PET SUVR, wherein each of pS199, pT175, and pS202 negatively correlates with tau PET SUVR.

12. A method for determining whether a human subject is likely to have Alzheimer's disease (AD), comprising
a. detecting the level of at least one tau phosphosite in a cerebrospinal fluid (CSF) sample from the subject comprising (a) one or more of pT111, pT153, and pT175, and optionally one or more of pT181, pS199, pS202, pT205, pS208, pS214, pT217, and pT231; (b) one or both of pS214 and pT231 and optionally one or more of pT111, pT153, pT175, pT181, pS199, pS202, pT205, pS208, and pT217; (c) pT217 and one or more of pT111, pT153, pT175, pS199, pS208, pS214, and pT231; or (d) four or more of pT111, pT153, pT181, pS208, pT217, and pT231; and
b. determining whether the subject is likely to have AD based on the level of the at least one tau phosphosite in the sample compared to levels of the phosphosite in control samples of prodromal AD, mild AD, moderate AD, and/or cognitively normal (CN) subjects, wherein the levels of pT175 and pS202 decrease with increasing AD stage, the levels of pT111, pT153, pT181, pS199, pS208, pS214, pT217 and pT231 are higher in AD subjects than in CN subjects, and the levels of pS214 and pT231 are higher in prodromal AD subjects than in CN subjects but lower in mild and moderate AD subjects than in prodromal AD subjects.

13. The method of claim 12, wherein the method is for determining whether the human subject is likely to have prodromal AD.

14. The method of claim 12, wherein the method is for determining whether the human subject is likely to have mild AD.

15. The method of claim 12, wherein the method is for determining whether the human subject is likely to have moderate AD.

16. A method of staging an Alzheimer's disease (AD) or potential AD subject as likely to be clinically normal (CN) or to have prodromal, mild, or moderate AD, comprising:
a. detecting the level of at least one tau phosphosite in a CSF sample from the subject comprising (a) one or more of pT111, pT153, and pT175, and optionally one or more of pT181, pS199, pS202, pT205, pS208, pS214, pT217, and pT231; (b) one or both of pS214 and pT231 and optionally one or more of pT111, pT153, pT175, pT181, pS199, pS202, pT205, pS208, and pT217; (c) pT217 and one or more of pT111, pT153, pT175, pS199, pS208, pS214, and pT231; or (d) four or more of pT111, pT153, pT181, pS208, pT217, and pT231; and
b. determining whether the subject is likely to have AD based on the level of the at least one tau phosphosite in the sample compared to levels of the phosphosite in control samples of prodromal AD, mild AD, moderate AD, and/or cognitively normal (CN) subjects, wherein levels of pT175 and pS202 decrease with increasing AD stage, levels of pT111, pT153, pT181, pS199, pS208, pS214, pT217 and pT231 are higher in AD subjects than in CN subjects, and levels of pS214 and pT231 are higher in prodromal AD subjects than in CN subjects but lower in mild and moderate AD subjects than in prodromal AD subjects.

17. The method of any one of claims 12-16, wherein the method further comprises correlating the level of the at least one tau phosphosite in the sample with a tau PET SUVR (standard uptake value rate) for the subject, such as a tau ([¹⁸F]GTP1) PET SUVR.

18. The method of claim 17, wherein the level of at least one of pT217, pS199, pT175, and pS202 is correlated with the tau PET SUVR, wherein pT217 positively correlates with tau PET SUVR, and wherein each of pS199, pT175, and pS202 negatively correlates with tau PET SUVR.

19. The method of any one of claims 1-18, wherein the subject:
a. Is known to be Aβ+, for example, based on an amyloid beta PET scan;
b. Has not received an amyloid beta PET scan;
c. Has not previously received a tau PET scan, such as a tau ([¹⁸F]GTP1) PET imaging scan; or
d. Has not previously received either an amyloid beta PET scan or a tau PET scan.

20. The method of any one of claims 1-19, wherein the level of at least one tau phosphosite in a CSF sample from the subject is detected by liquid chromatography mass spectrometry (e.g. tandem mass spectrometry) analysis (LC-MS or LC-MS/MS)).

21. The method of any one of claims 1-20, wherein CN subjects comprise Aβ+ but CN subjects, Aβ− CN subjects, or all CN subjects regardless of Aβ status.

22. The method of any one of claims 1-21, comprising detecting the level of one or more of pT111, pT153, or pT175.

23. The method of claim 22, wherein the method comprises determining whether the level of pT111 and/or pT153 is elevated in comparison to CN subjects, and/or wherein the method comprises determining whether the level of pT175 is reduced in comparison to CN subjects and/or in comparison to prodromal or mild AD subjects.

24. The method of any one of claims 1-23, comprising detecting the level of two or more of pT111, pT153, pT205, pS208, pS199, and pT217.

25. The method of any one of claims 1-24, wherein the method comprises detecting the level of one or more of pT181, pS214, and pT231, and determining whether the level of pT181, pS214, and/or pT231 is elevated in comparison to CN subjects or is elevated in comparison to mild or moderate AD subjects.

26. The method of claim 25, comprising detecting the level of two or more of pT181, pS214, and pT231, or all three of pT181, pS214, and pT231.

27. The method of claim 25 or 26, wherein the method comprises detecting the level of pS214, and determining whether the level of pS214 is elevated in comparison to CN subjects or is elevated in comparison to mild or moderate AD subjects.

28. The method of any one of claims 1-27, wherein the method comprises detecting the level of one or both of pT175 and pS202, and determining whether the level of pT175 and/or pS202 is reduced in comparison to CN subjects and/or is elevated in comparison to mild or moderate AD subjects.

29. The method of any one of claims 1-28, wherein the method comprises detecting the level of no more than 11 phosphosites.

30. The method of claim 29, wherein the method comprises detecting the level of no more than 8 phosphosites.

31. The method of claim 29, wherein the method comprises detecting the level of no more than 6 phosphosites.

32. The method of claim 29, wherein the method comprises detecting the level of no more than 4 phosphosites.

33. The method of any one of claims 1-32, wherein the method comprises detecting the level of one or more of pT111, pT153, and pT175, and additionally one or more of pT181, pS199, pS202, pT205, pS208, pS214, pT217, and pT231.

34. The method of any one of claims 1-33, wherein the method comprises detecting the level of one or both of pT175 and pS202.

35. The method of any one of claims 1-34, wherein the method comprises detecting the level of one or both of pS214 and pT231 and additionally one or more of pT111, pT153, pT175, pT181, pS199, pS202, pT205, pS208, and pT217.

36. The method of any one of claims 1-35, wherein the method comprises detecting the level of pT217 and one or more of pT111, pT153, pT175, pS199, pS208, pS214, and pT231.

37. The method of any one of claims 1-36, wherein the method comprises detecting the level of each of pT217, pT153, pT181, pT111, pS208, and pT231.

38. The method of any one of claims 1-37, wherein the level of the phosphosite(s) is calculated as the ratio of the level of the phosphorylated site to the level of its corresponding unposphorylated site (e.g., by calculating the ratio of the level of a peptide comprising the phosphorylated site to the level of the corresponding unphosphorylated peptide in a MS analysis).

39. The method of any one of claims 12-16 or 19-38, wherein the subject has not previously had a tau PET and/or amyloid beta PET and wherein the method is used to determine whether the subject should receive a tau PET and/or amyloid beta PET.

40. The method of any one of claims 1-39, further comprising performing one or more of tau PET, amyloid PET, MRI, pTau181 ELISA, and cognitive function assays on the subject.

41. A method of treating a subject with an anti-tau therapy, wherein the subject has been tested prior to treatment according to the method of any one of claims 1-40 and found to have a level of tau phosphosite(s) corresponding to that of an AD subject (e.g., a prodromal, mild, or moderate AD subject).

42. A method of treating a subject with an anti-tau therapy, wherein the subject has been tested prior to treatment according to the method of any one of claims 1-40, the method comprising determining that the subject has a level of tau phosphosite(s) corresponding to that of an AD subject (e.g., a prodromal, mild, or moderate AD subject), and subsequently administering an anti-tau therapy to the subject.

43. The method of claim 41 or 42, wherein the anti-tau therapy comprises an anti-tau antibody.

44. The method of claim 43, wherein the anti-tau antibody is gosuranemab, ABBV-8E12, zagotenemab, or semorinemab.

45. The method of claim 43, wherein the anti-tau antibody is semorinemab.

46. The method of any one of claims 41-45, wherein the subject is re-tested according to the method of any one of any one of claims 1-40 following administration of the anti-tau therapy.
